(19) United States
(12) Patent Application Publication
(10) Pub. No.: US 2026/0335508 A1
(43) Pub. Date: Oct. 29, 2026

(54) SYSTEMS AND METHODS FOR SURFACE MODIFICATION OF IMPLANTABLE DEVICES

(71) Applicant: Warsaw Orthopedic, Inc., Warsaw, IN (US)

(72) Inventors: Michelle B. Gallagher, Fuquay-Varina, NC (US); James T. Sugar, Wauwatosa, WI (US); Yana Rawinski, Wauwatosa, WI (US); Michael Vidra, Export, PA (US); Jordan Incerpi, Pittsburgh, PA (US)

(21) Appl. No.: 19/658,966

(22) Filed: Apr. 27, 2026

Related U.S. Application Data
(60) Provisional application No. 63/796,048, filed on Apr. 28, 2025.

(51) Int. Cl. A61L 27/50

(52) U.S. Cl. CPC A61L 27/50; A61L 2400/18; A61L 2430/02

(57) ABSTRACT

A method for modifying a surface of an implantable device is provided. The method including contacting the implantable device with an alkaline etch solution including a chelating agent. The alkaline etch solution etches an alpha phase and a beta phase of the surface of the implantable device. The implantable device is contacted with a hydrogen peroxide solution. The hydrogen peroxide solution further etches the beta phase of the surface of the implantable device.

### CROSS-REFERENCE TO RELATED APPLICATION

This application claims the benefit of and priority to U.S. Provisional Patent Application No. 63/796,048 filed Apr. 28, 2025, the entire disclosure of which is incorporated by reference herein.

### TECHNICAL FIELD

The present disclosure relates generally to the field of implantable devices. In particular, the present disclosure relates to modifying surfaces of implantable devices to form microscale structures and nanoscale structures to facilitate bone growth.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In clinical use, a variety of conditions may warrant repair and/or replacement of an internal body part, such as bone. For example, orthopedic implants may be used to correct skeletal defects. Integration of orthopedic implants with adjacent bone may be challenging due to mechanical, chemical, and/or biochemical interactions between bone-contacting surfaces of the orthopedic implants and adjacent bone. For example, naturally continuous bone formation may be based on a process of osteoblasts forming new bone in small depressions generated by osteoclasts upon breaking down of old bone tissue. Previously available orthopedic implants may use line-of-site manufacturing techniques to generate surfaces of the orthopedic implants to mimic natural occurring bone surfaces. However, line-of-site manufacturing techniques limit large-scale processing of surfaces of controlled surface topography and involve customization of surface modification processes based on implant properties. Accordingly, new technologies for surface modification of orthopedic implants for osseointegration are needed.

### SUMMARY

Certain embodiments are summarized below. These embodiments are not intended to limit the scope of the disclosure. Indeed, the present disclosure may encompass a variety of forms that may be similar to or different from the embodiments set forth below. The implantable device of the current application will address the issues discussed prior, and further include improved surface modification techniques for improved osseointegration.

In one embodiment, a method for modifying a surface of an implantable device is provided. The method including contacting the implantable device with an alkaline etch solution including a chelating agent. The alkaline etch solution etches an alpha phase and a beta phase of the surface of the implantable device. The implantable device is contacted with a hydrogen peroxide solution. The hydrogen peroxide solution further etches the beta phase of the surface of the implantable device.

In one embodiment, a method for forming one or more microstructures, one or more nanostructures, or a combination thereof on a surface of an implantable device is provided. The method including etching an alpha phase and a beta phase of the surface of the implantable device and etching the beta phase of the surface of the implantable device. Further etching the beta phase of the surface of the implantable device comprises reducing a vanadium content of the etched surface of the implantable device.

In one embodiment, a method for modifying a surface of an implantable device is provided. The method including contacting the implantable device with an alkaline etch solution under a first set of reaction conditions to form an alkaline etched implantable device. The alkaline etch solution comprises a metal hydroxide and a chelating agent. The alkaline etch solution etches an alpha phase and a beta phase of the surface of the implantable device. The method also including contacting the alkaline etched implantable device with an acid solution to form an acid treated implant and contacting the acid etched implantable device with a hydrogen peroxide solution under a second set of reaction conditions to form a peroxide etched implantable device. The hydrogen peroxide solution etches the beta phase of the surface of the implantable device. The first set of reaction conditions and the second set of reaction conditions are different from one another.

### BRIEF DESCRIPTION OF THE DRAWINGS

Advantages of the disclosed techniques may become apparent upon reading the following detailed description and upon reference to the drawings in which:

FIG. 1 is a block diagram of a process to modify a surface of one or more implants to produce one or more modified implants, in accordance with an embodiment of the present disclosure;

FIG. 2 is a flow diagram of a process to etch an α-phase and/or a β-phase of a surface of one or more implant to produce one or more modified implants, in accordance with an embodiment of the present disclosure;

FIG. 3A is a scanning electron image (SEM) image of a portion of a modified implant having a plurality of microscale structures generated by contacting an implant with an alkaline solution and a peroxide solution, in accordance with an embodiment of the present disclosure;

FIG. 3B is a SEM image of a portion of the modified implant of FIG. 3A, including microscale structures and nanoscale structures generated by contacting the implant with the alkaline solution and the peroxide solution, in accordance with an embodiment of the present disclosure;

FIG. 3C is a SEM image of a portion of the modified implant of FIG. 3A, including nanoscale structures generated by contacting the implant with the alkaline solution and the peroxide solution, in accordance with an embodiment of the present disclosure;

FIG. 4A is a SEM image of a portion of an alkaline etched implant, in accordance with an embodiment of the present disclosure;

FIG. 4B is a SEM image, including microscale structures and nanoscale structures on a portion of a surface of an alkaline etched implant, in accordance with an embodiment of the present disclosure;

FIG. 4C is a SEM image of a portion of the modified implant of FIG. 4B at an increased magnification, including the plurality of nanoscale features on the portion of the surface of the alkaline etched implant of FIG. 4B, in accordance with an embodiment of the present disclosure;

FIG. 5A is a SEM image of a portion of a modified implant including microscale structures and nanoscale porous structures, in accordance with an embodiment of the present disclosure;

FIG. 5B is a SEM image of a portion of the modified implant of FIG. 5A at an increased magnification, including microscale structures and nanoscale porous structures, in accordance with an embodiment of the present disclosure;

FIG. 5C is a SEM image of a portion of the modified implant of FIG. 5B at an increased magnification, including nanoscale porous structures, in accordance with an embodiment of the present disclosure;

FIG. 6 is a transmission electron microscopy (TEM) image of a modified implant including oxide formation, in accordance with an embodiment of the present disclosure;

FIG. 7A is a first embodiment of a high resolution TEM (HR-TEM) image of a modified implant showing an amorphous lattice of the modified implant and formation of an oxide layer, in accordance with an embodiment of the present disclosure;

FIG. 7B is a second embodiment of a high resolution TEM (HR-TEM) image of a modified implant showing an amorphous lattice of the modified implant and formation of a surface oxide, in accordance with an embodiment of the present disclosure;

FIG. 8A is an electron dispersive spectroscopy (EDS) graph of a portion of the modified implant illustrating formation of vanadium oxide, including an inset EDS map of the TEM image of a correlated measurement area of the modified implant, in accordance with an embodiment of the present disclosure;

FIG. 8B is Raman graph of the portion of the modified implant of FIG. 8A, in accordance with an embodiment of the present disclosure;

FIG. 9A is an EDS graph of a portion of the modified implant illustrating formation of aluminum oxide, including an inset EDS map of the TEM image of a correlated measurement area of the modified implant, in accordance with an embodiment of the present disclosure;

FIG. 9B is Raman graph of the portion of the modified implant of FIG. 9A, in accordance with an embodiment of the present disclosure;

FIG. 10A is an EDS graph of a portion of the modified implant illustrating formation of aluminum oxide and vanadium oxide, including an inset EDS map of the TEM image of a correlated measurement area of the modified implant, in accordance with an embodiment of the present disclosure;

FIG. 10B is Raman graph of the portion of the modified implant of FIG. 10A, in accordance with an embodiment of the present disclosure;

FIG. 11A is an EDS graph of a portion of the modified implant illustrating formation of titanium oxide and vanadium oxide, including an inset EDS map of the TEM image of a correlated measurement area of the modified implant, in accordance with an embodiment of the present disclosure;

FIG. 11B is Raman graph of the portion of the modified implant of FIG. 11A, in accordance with an embodiment of the present disclosure;

FIG. 12 is a graph of metal to oxide ratios of implants and treated implants measured using X-ray photoelectron spectroscopy (XPS), in accordance with an embodiment of the present disclosure;

FIG. 13A is a SEM image of a portion of a modified implant generated using additive manufacturing, including a plurality of microscale structures generated by contacting an implant with an alkaline solution and a peroxide solution, in accordance with an embodiment of the present disclosure;

FIG. 13B is a SEM image of a portion of the modified implant of FIG. 13A, including microscale structures and nanoscale structures generated by contacting the additively manufactured implant with the alkaline solution and the peroxide solution, in accordance with an embodiment of the present disclosure;

FIG. 13C is a SEM image of a portion of the modified implant of FIG. 13B at a higher magnification, including nanoscale structures generated by contacting the modified additively manufactured implant with the alkaline solution and the peroxide solution, in accordance with an embodiment of the present disclosure;

FIG. 14 is a HR-TEM image of the modified additively manufactured implant including nanoscale structures and oxide formation, in accordance with an embodiment of the present disclosure;

FIG. 15A is a high resolution TEM (HR-TEM) image of an additively manufactured implant including one or more nanocrystals within an amorphous lattice of the oxide layer, in accordance with an embodiment of the present disclosure;

FIG. 15B is a diffraction pattern of the HR-TEM image of FIG. 15A at a face centered cubic (FCC) 110 zone axis indicative of short-range ordering due to the presence of the nanocrystals within the additively manufactured implant, in accordance with an embodiment of the present disclosure;

FIG. 16A is a HR-TEM image including one or more EDS imaging areas of a modified additively manufactured implant, in accordance with an embodiment of the present disclosure;

FIG. 16B is a HR-TEM image including one or more EDS imaging areas of a modified additively manufactured implant at an increased magnification, in accordance with an embodiment of the present disclosure;

FIG. 17 is a graph including crystal violet absorbance to characterize cell numbers on the modified implant in comparison to comparative samples, in accordance with an embodiment of the present disclosure;

FIG. 18 is a graph of alkaline phosphate (ALP) expression in a modified implant in comparison to comparative samples, in accordance with an embodiment of the present disclosure;

FIG. 19 is a graph of osteocalcin (OCN) expression of a modified implant in comparison to comparative samples, in accordance with an embodiment of the present disclosure;

FIG. 20 is a graph of osteopontin (OPN) in a modified implant in comparison to comparative sample, in accordance with an embodiment of the present disclosure;

FIG. 21 is a graph of sclerostin (SOST) expression of a modified implant in comparison to comparative samples, in accordance with an embodiment of the present disclosure;

FIG. 22 is a graph of mineralization in a modified implant in comparison to comparative samples, in accordance with an embodiment of the present disclosure;

FIG. 23 is a graph of osteocalcin (OCN) expression of a wrought modified implant and an additively manufactured modified implant in comparison to comparative samples, in accordance with an embodiment of the present disclosure; and

FIG. 24 is a graph of osteopontin (OPN) expression of a wrought and of an additive modified implant in comparison to comparative samples, in accordance with an embodiment of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The methods discussed herein include various steps represented by blocks in flow diagrams. It should be noted that at least some steps may be performed as an automated procedure by one or more components of a system. Although the flow diagrams may illustrate the steps in a certain sequence, it should be understood that the steps may be performed in any suitable order and certain steps may be carried out simultaneously, where appropriate. Additionally, steps may be added to or omitted from of the methods.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. All numerical values within the detailed description herein are modified by “about” the indicated value, and take into account experimental error, manufacturing tolerances, and variations that would be expected by a person having ordinary skill in the art. For example, “about” or “approximately” may refer to ±0.2%, ±0.5%, ±1%, ±2, ±5%, ±10%, or ±15%. All ranges expressed herein should include both end points as two specific embodiments unless specified or indicated to the contrary.

As used herein, the term “nanoscale” refers to a material having at least one dimension less than 100 nanometers (nm). As used herein, the term “microscale” refers to material having a least one dimension between 100 nm and 1000 micrometers (μm).

Implantable devices (e.g., implants) may be used for bone replacement such as intervertebral cages, endosseous implants, artificial knees, and the like. Such implantable devices may be fabricated from titanium as titanium has been shown to exhibit biocompatibility and mechanical properties suitable for use in bone replacement. Titanium surfaces have shown direct integration with bone; however it is presently recognized that increased osseointegration may be facilitated by modification in surface roughness, surface chemistry, surface energy, or a combination thereof. Within the context of surface modification of implantable devices, multi-level roughness (i.e., roughness features provided at different size or geometric scales) may offer improved integration of implantable devices for bone replacement. However, current techniques (e.g., shot peening, grit blasting, laser ablation, etc.) remove measurable amounts of material from surfaces of implantable devices during surface modification and may produce roughness on a macroscale that may degrade performance and/or impact strength of implantable devices. As such, new technologies for surface modification of implantable devices offering multi-level roughness with lower material loss for improved osseointegration are needed.

Systems and methods disclosed herein relate to surface modification of bone-contacting implantable devices (e.g., implant bodies) to provide modified implantable devices to improve osseointegration. In this manner, modified implantable devices may include one or more bone-contacting surfaces and/or non-bone-contacting surfaces with both engineered (as opposed to incidental) microstructures (e.g., micrometer-scale structures) and nanostructures (nanometer-scale structures) to promote expression of osteogenic markers. Surface modification of implantable devices with microstructures and nanostructures promote integration with adjacent bone by mimicking the hierarchical structure of natural occurring bone surfaces generated when osteoclasts break down old bone tissue. The microstructures and nanostructures engineered on the implantable devices provide texture to serve as a scaffold to increase osteogenic potential of fixation to adjacent bone. It should be noted, texturing of the implantable devices may occur on both the bone-contacting and non-bone-contacting surfaces of modified implantable devices. Implantation of the modified implantable device may induce bone growth along one or more surfaces (e.g., bone-contacting and/or non-bone contacting surfaces) of the implantable devices, as such initially non-bone-contacting surfaces may become bone-contacting surfaces of the implantable device after a period of time. As such, texturing one or more non-bone contacting surfaces of the implantable devices may be beneficial to promote osseointegration. As described herein, surface modification of the implantable devices may be achieved by treating surfaces of the implantable devices with subtractive surface modification techniques. The subtractive surface modification techniques include an alkaline etch treatment process to provide control of surface modification based on chemical treatment of one or more surfaces of the bone-contacting implantable devices. Accordingly, as compared with previous techniques, the embodiments disclosed herein provide techniques to enable scalable production (e.g., improved manufacturing throughput) of bone-contacting implant surfaces with controlled surface topography with improved osseointegration.

In some embodiments, bone-contacting implantable devices may be modified by applying a surface treatment process. The surface treatment process may include an alkaline etch treatment, an acid flash, a hydrogen peroxide treatment, one or more cleaning steps, or a combination thereof. For example, the surfaces of the implantable device may be contacted with an alkaline etch solution to etch an α-phase and/or a β-phase of the surface of the implantable device. In some instances, the alkaline etched surfaces may be treated with a nitric acid solution to remove one or more alloying elements from the alkaline etched surfaces of the implantable device. The alkaline etched surfaces of the implantable device may be treated with a hydrogen peroxide solution. The hydrogen peroxide solution may etch the β-phase of the surface of the implantable device. In certain embodiments, treatment with the hydrogen peroxide solution may reduce a vanadium content of the surfaces of the implantable device. The surface treatment process may form both microstructures and nanostructures on the surfaces of the implantable device.

In some embodiments, the surfaces of the implantable device may offer roughened surface topography for improved osseointegration. The surface topography of the bone-contacting implantable device as a result of the surface treatment process may include surface modification on the microscale and nanoscale. As will be described herein, the surface topography of the implantable device may include microscale features with a roughness ranging between approximately 0.1 μm and 10.0 μm. Formation of the surface topography may result from a faster removal of the alpha phase as compared to removal of the beta phase of the implantable device. Removal of the alpha phase may expose the beta grains at or extended beyond a surface of the implantable device. “Roughness” as described herein is measured in terms of the average height between peaks and valleys of microscale features or nanoscale features. Further, formation of nanoscale features with a roughness ranging between approximately 1 nm and 100 nm may result from surface modification of α-grains, formation of nanoscale features on microscale beta-grain features, or a combination thereof.

In certain embodiments, the microscale features and the nanoscale features may include complex heterogenous surface oxides. As such, a heterogenous surface oxide present on the one or more bone-contacting and/or non-bone-contacting surfaces may include aluminum oxide, titanium oxide, vanadium oxide, or a combination thereof. For example, surfaces of titanium based implantable devices may be formed with bone-contacting surfaces including a vanadium to titanium ratio greater than or equal to approximately 0.02. Without wishing to be bound by theory, it is believed that the heterogenous surface oxides may improve biocompatibility and/or reduce corrosion of the bone-contacting implantable devices. Without wishing to be bound by theory, the presence of the heterogenous surface oxides may improve osseointegration by altering protein adsorption and/or configuration as compared to untreated surfaces. Further, in some embodiments, the microscale features and the nanoscale features may offer a surface hierarchal structure to improve osteogenic activity while offering low levels of friction with adjacent bone structures.

In some embodiments, the implantable devices may be formed using additive manufacturing. Additive manufacturing may include three dimensional (3D) printing of implantable devices using metal powders. Additive manufacturing of implantable devices may be used to produce customized implants for use in bone replacement. The additive manufactured implantable device may be treated with the surface treatment process to form microstructures and nanostructures to improve osseointegration. Due to the non-equilibrium conditions present during metal additive manufacturing, one or more treated surfaces of the additive manufactured implantable device may include surface roughness, surface chemistry, surface energy, or a combination thereof differing from modified implantable devices formed through traditional processes (e.g., powder metallurgy, mechanical alloying, etc.). Implantable devices may include a hexagonal close-packed (HCP) phase corresponding to an α-phase and a body-centered cubic (BCC) phase corresponding to a β-phase of the modified additive manufactured implants. In some embodiments, modification of additive manufactured implants may lead to formation of one or more additional crystal phases. For example, a face-centered-cubic (FCC) phase including one or more nanocrystals may be formed in additive manufactured implantable devices. As such, nanocrystals with FCC character may be formed during modification of additive manufactured devices forming FCC nanocrystals within an HCP phase and/or a BCC. Without wishing to be bound by theory, it is believed that the FCC phase may result in changes in osseointegration due to differences in the crystal lattice at surfaces of the modified additive manufactured implants and may offer increased mechanical strength.

In some embodiments, modified implantable devices (e.g., modified wrought implants and/or modified additive manufactured implants) may provide bone-contacting and/or non-bone-contacting surfaces with microfeatures and nanofeatures for improved osseointegration as indicated by increased osteogenic differentiation of stem cells or osteoblast cell types. Osteogenic differentiation is a continuous process characterized by a rise and fall of several proteins such as alkaline phosphatase (ALP) and/or osteocalcin (OCN). Additional proteins expressed represent lifetime osteogenic markers, such as osteopontin (OPN). Expression of lifetime osteogenic markers continue to increase with time as cells mature to an osteoblast phenotype. Osteoblast differentiation begins with mesenchymal stem cells (MSCs) progressing to an intermediate progenitor capable of undergoing either osteogenesis or chondrogenesis and expressing ALP. Intermediate progenitors that commit to an osteogenic lineage, now termed preosteoblasts, increase expression of ALP, and start expressing the extracellular matrix protein OPN. As the preosteoblast progresses to an osteoblast expression of OPN continues to increase and expression of OCN is increased. The osteoblast will in time mature further and begin transitioning to an osteocyte or undergo apoptosis. A mature osteoblast state is characterized by a decrease in ALP. Once the osteoblast differentiates to an osteocyte the expression of OCN is decreased. OPN expression is detected throughout cell lifetime from a preosteoblast to an osteocyte. Finally, sclerostin (SOST) is a secreted protein that regulates bone remodeling and is produced by a terminally differentiated osteocyte. The proteins expressed by osteoblasts work in concert to produce an osteoid matrix of Type I collagen network. The osteoid matrix of Type I collagen within the presence of increased phosphate ions mineralizes. Without wishing to be bound by theory, such mineralization may be catalyzed by an early bone marker ALP. Mineralization of newly synthesized osteoid deposited by the osteoblasts leads to bone tissue formation. Surface texture and/or chemistry are known to influence one or multiple steps in the osteoblast differentiation and mineralization process. For example, the surface texture of the modified implantable devices (e.g., one or more representative test discs) may show an increase in a number of MSCs present within the first 48 hours of seeding cells when compared to a machined only finish and/or one or more additional surface modification methods such as grit blasting followed by acid etching. Cells on one or more modified surfaces, similar to other nano surface modifications, may show improved expression of ALP and OCN by stem cells (MSCs) at early timescales (e.g., seven days, fourteen days) as compared to machined only samples. Expression of ALP and OCN by MSCs in contact with the modified surfaces is indicative of increased osteoblast expression during bone formation. Such increased osteoblast expression during bone formation may improve osseointegration. Additionally and/or alternatives, as disclosed herein, the modified surfaces of the modified implantable device may support improvement in bone formation as indicated by an increased osteopontin (OPN) expression by MSCs over a timecourse, indicating potential for active bone formation at bone-contacting surfaces of the modified implantable devices. Further, modified implantable devices may show improved expression of SOST by MSCs, a late-differentiation marker, at later timescales (e.g., 14 days and/or 21 days) as compared to samples with other surface finishes (e.g., comparative samples) indicating differentiation of stem cells to mature bone cells needed for osseointegration of the modified implantable device. Bone formation may be supported by an increase in the percent of surface area mineralized with discrete nodules measured on the surfaces of modified implantable devices compared with machined only surfaces. As such, surface topography of the modified implantable devices including microfeatures and nanofeatures may support bone growth at increased levels as compared to alternative implants with roughened, smooth, and/or machined surfaces. In this manner, surface modification techniques disclosed herein may produce implantable devices with surfaces that promote improved osseointegration.

### Modification of Bone-Contacting Surfaces of Implantable Devices

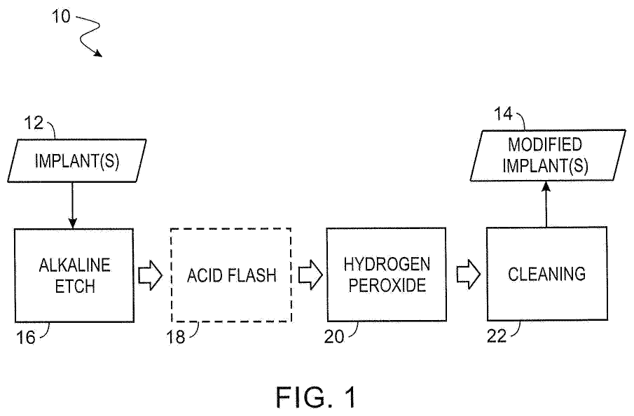

FIG. 1 is a block diagram of a process 10 to modify a surface of one or more implants 12 to produce one or more modified implants 14, in accordance with an embodiment of the present disclosure. The one or more implants 12 may be treated in one or more stages. It should be noted that the illustrated stages are provided as examples and more, fewer, or different stages may be included in the process 10. As shown, the process 10 may include an alkaline etch stage 16, an acid flash stage 18, a hydrogen peroxide stage 20, and a cleaning stage 22. It should be noted, the implants 12 may be treated with one or more finishing steps prior to the process 10. For example, the implants 12 may be treating with one or more procedures such as machining, anodizing, blasting, and the like.

The process 10 may be used to modify the surface of the implants 12. In some embodiments, one or more portions of the surface of the implants 12 may be chemically etched. Chemical etching of the implants 12 may preferentially etch an α-phase or a β-phase of the surface of the implant 12. The illustrated stages may be used in combination to treat the α-phase and/or β-phase of the surface of the implants 12 and remove one or more alloying elements from the surface of the implants 12. It should be noted, that a treated (e.g., chemically etched) surface and/or treated portions of the surface of the implants 12 correspond to one or more bone-contacting surfaces and/or non-bone-contacting surfaces of the implants 12 (e.g., one or more implantable devices). As such, the treated portions of the surface of the implants 12 may be formed by the process 10 generating one or more microstructures, one or more nanostructures, or a combination thereof on the surface of the implants 12.

The alkaline etch stage 16 may be initiated after receiving the one or more implants 12. The implants 12 may be orthopedic implantable devices that may be used to correct skeletal defects. The implants 12 (e.g., implant body) may be formed from vanadium, titanium, titanium alloy, alloys of titanium and nickel, alloys of titanium and zirconium, aluminum, aluminum alloy, alloys of titanium and aluminum, stainless steel (e.g., surgical grade steel), magnesium alloys, alloys of cobalt and chromium, nitinol, tantalum composites, metal powders (e.g., additive manufacturing), ceramics, and the like. In an embodiment, the implants 12 may be formed through additive manufacturing. Additive manufacturing techniques may include electron beam melting, selective laser sintering, direct metal laser sintering, fused deposition modeling, direct metal deposition, laser engineered net shaping, wire-based directed energy deposition, and the like.

The alkaline etch stage 16 may include treating the surface of the implants 12 in an alkaline solution. The alkaline solution may include a base, an oxidizing agent, one or more chelating agent, one or more amines, or a combination thereof. In an embodiment, the base may include a metal hydroxide (e.g., sodium hydroxide, potassium hydroxide) or one or more additional suitable strong bases. The oxidizing agent may include hydrogen peroxide, ozone, or a combination thereof. It should be noted, in some embodiment the alkaline solution may not include the oxidizing agent. That is, the alkaline solution may consist essentially of the base and the chelating agent. The chelating agent may include gluconic acid, sodium gluconate or other gluconate salt, ethylenediaminetetraacetic (EDTA) acid, nitrilotriacetic acid, or one or more additional chelating agents. The chelating agent may be provided in salt or acid form. The chelating agent may selectively bind to one or more metal ions present in one or more portions of the surface of the implants 12 during the alkaline etch stage 16. As such, the chelating agent may modify the surface of the implants 12 by increasing the etching rate of the α-phase and/or β-phase of the surface of the implants 12. The amines may include one or more alkanolamines. The amines may be used to control the pH of the alkaline solution by increasing an amount of free ammonia available to etch the implants 12. In an embodiment, the pH of the alkaline solution may range from 7.0 to 12.0, 7.0 to 9.0, 8.0 to 12, 8.5 to 11.0, or 9.0 to 11.0. In some embodiments, the alkaline solution may include 5 to 75 wt %, 5 to 50 wt %, 10 to 35 wt %, or 18 to 30 wt %, or 22 to 25 wt %, of the base, 0 to 5 wt %, 5.5 to 10.5 wt %, or 6 to 7 wt % of the oxidizing agent, 0.1 to 40 wt %, 2 to 8 wt %, 3 to 6 wt %, or 3.5 wt % to 5.5 wt % of the chelating agent, 0 to 40 wt %, 2 to 10 wt % of the amine, or a combination thereof.

In some embodiments, the implants 12 may be treated by the alkaline etch stage 16 of the process 10 at various temperature and times. In practice, the temperature of the alkaline solution may affect an etch rate of the surface of the implants 12. As such, the temperature of the alkaline solution may increase or decrease during the alkaline etch stage 16 to increase or decrease the etching rate. In an embodiment, the temperature of the alkaline solution may range from 15 °C to 140 °C, 20 °C to 100 °C, 30 °C to 100 °C, 45 °C to 100 °C, 65 °C to 100 °C, 75 °C to 90 °C, 85 °C to 95 °C, 80 °C to 95 °C, 92 °C to 97 °C, 88 °C to 94 °C, and the like. The implants 12 may be treated in the alkaline etch stage 16 for a period of time based on a desired etch rate of the surface of the implants 12. The period of time may range from 1 minute (min) to 100 hours, 1 min to 72 hours, 1 min to 48 hours, 1 min to 24 hours, 1 min to 60 mins, 5 mins to 30 mins, 15 mins to 30 mins, 20 mins to 30 mins, 20 mins to 25 mins, and the like. In an embodiment, an etch rate of the implants 12 may range from 0.2 to 1.5 mils (e.g., 1 mil is 0.001 inches) per 60 mins. The etch rate may range from 0.3 to 1.5 mils per 60 mins, 0.3 to 1.47 mils per 60 mins, and the like. In any case, the temperature and/or time of the alkaline etch stage 16 may be adjusted to control the amount of the etching by the alkaline solution.

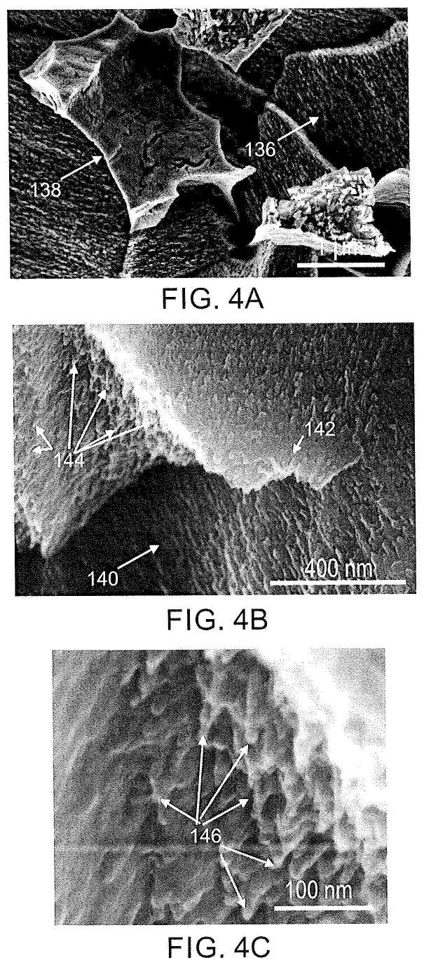

In certain embodiments, the alkaline etch stage 16 may etch portions of the surface of the implants 12 to generate one or more alkaline etched implants. The alkaline etched implants may include microstructure and nanostructure surfaces discussed below in reference to FIGS. 4A-4C. As the implants 12 are etched one or more alloying elements (e.g., components etched from the surface of the implants 12) may be formed as insoluble precipitates and/or soluble salts. In some instances, the insoluble precipitates may be located on the surface of the alkaline etched implants.

After the alkaline etch stage 16, the process 10 may advance to the acid flash stage 18. The acid flash stage 18 may be used to remove the alloying elements (e.g., such as insoluble precipitates, free iron, one or more additional metal precipitates). Additionally and/or alternatively, the acid flash stage 18 may passivate the surface (e.g., remove free metal ions and/or generate one or more oxide layers) of the alkaline etched implants. It should be noted, in some embodiments the acid flash stage 18 may be omitted from the process 10.

The acid flash stage 18 generally includes treating the implant (e.g., after the alkaline etch stage 16) with an acid solution. The acid solution may include nitric acid, citric acid, and/or one or more additional components (e.g., sodium dichromate, etc.). The acid solution may be an aqueous solution with acid (e.g., nitric acid, citric acid) ranging from 5 to 60% by volume, 5 to 15% by volume, 5 to 10% by volume, 10 to 15% by volume, and the like. The acid solution may be heated during treatment of the alkaline etched implants in the acid flash stage 18. For example, the acid solution may be heated to a temperature ranging from 10 °C to 50 °C, 18 °C to 41 °C, 20 °C to 40 °C, 25 °C to 32 °C, room temperature (e.g., about 20 °C), and the like. The alkaline etched implants may be treated in the acid solution for a period of time to form one or more acid treated implants. The period of time may range from 1 sec to 5 mins, 1 sec to 90 secs, 1 sec to 60 secs, 10 secs to 90 secs, 60 secs to 120 secs, and the like. The acid flash stage may be used to remove the alloying elements to reduce risk of dislodgement of alloyed components from the surface of the modified implants 14.

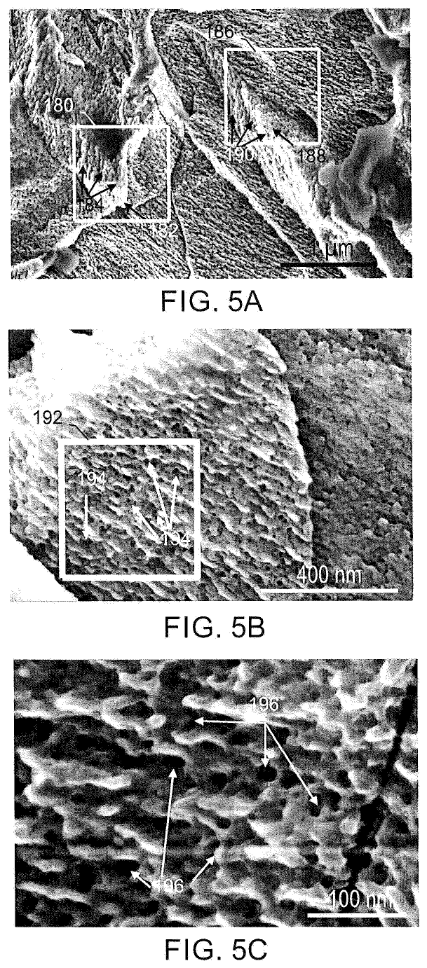

The process 10 may advance from the acid flash stage 18 (if performed) to the hydrogen peroxide stage 20. The hydrogen peroxide stage 20 may be used to further treat the acid treated implants by selectively removing a part of the β-phase surface structures thereby decreasing surface vanadium levels and optionally forming one or more additional nanofeatures. The hydrogen peroxide stage 20 generally includes etching the surface of the acid treated implants in a peroxide solution to form one or more peroxide etched implants. The peroxide solution may include 2 to 20% by volume, 5 to 15% by volume, 3 to 7% by volume, 5 to 10% by volume, 5 to 12% by volume, 6 to 9% by volume, or 7 to 8% by volume, 5 to 6% by volume, 6 to 8% by volume, 6 to 7% by volume hydrogen peroxide. It is presently recognized that adjusting the concentration of the peroxide solution may be used to tune the phase where the nanofeatures are formed. For example, treatment of the acid treated implants with the peroxide solution with an increased amount of hydrogen peroxide (e.g., 15-20% by volume) may form nanofeatures with nanoporous surface morphologies on the alpha phase. As another non-limiting example, treatment of the acid treated implants with the peroxide solution with a hydrogen peroxide volume ranging from 5 to 10% by volume may form nanofeatures on the beta phase of the peroxide etched implants without substantial change to the alpha phase of the peroxide etched implant. The peroxide solution may be heated to a temperature ranging from 35 °C to 60 °C, 37 °C to 53 °C, 40 °C to 55 °C, 44 °C to 53 °C, 47 °C to 50 °C and the like. The acid treated implants may be treated in the peroxide solution for a period of time to form the peroxide etched implants. The period of time may range from 1 min to 20 mins, 1 min to 15 mins, 10 mins to 20 mins, 10 mins to 15 mins, 13 mins to 17 mins, 14 mins to 16 mins and the like. Without wishing to be bound by theory, it is believed that treatment of the acid treated implants and/or the alkaline etched implants with the peroxide solution may reduce leaching of one or more components from the surfaces of the treated implants. For example, treatment with the peroxide solution reduces leaching of vanadium from the surfaces of the treated implants. In this manner, the hydrogen peroxide stage 20 may be used to selectively etch one or more phases of the acid treated implants. For example, the β-phase of the treated implants may be selectively etched by the peroxide solution. It should be noted, a concentration of hydrogen peroxide in the peroxide solution, a temperature of the peroxide solution, and/or a treatment time (e.g., period of time the implants 12 are in the peroxide solution) may be used to control selective etching of a particular phase of the alkaline etched implants and/or the acid treated implants. In some embodiments, the particular phase etched by the peroxide treatment may be based on a character of a material of the implants 12. For example, as discussed below in reference to FIG. 5A and FIG. 5B, increasing the concentration of the hydrogen peroxide in the peroxide solution may form nanopores in the surface of microfeatures and nanofeatures of the surface of the peroxide treated implant.

The process 10 may advance from the hydrogen peroxide stage 20 to the cleaning stage 22. The cleaning stage 22 may be used to clean the peroxide etched implants. The cleaning stage 22 may be used to remove components (e.g., such as insoluble precipitates, soluble salts, metal precipitates, and the like) from the peroxide etched implants to provide the modified implants 14. The cleaning stage 22 may include a series of rinsing steps to remove components not physically bound to the peroxide etched implants. In some embodiments, the cleaning stage 22 may include rinsing the peroxide etched implants in a deionized water bath. In some embodiments, one or more sonication cleaning steps may be used to physically dislodge any components not physically bound to the peroxide etched implants. It should be noted, one or more additional cleaning stages may be performed prior to and/or subsequent to the one or more illustrated stages 16, 18, 20. The one or more additional cleaning stages may remove any non-bound components from the surface of the implants 12 between treatments with the alkaline solution, the acid solution, and/or the peroxide solution.

In some embodiments, the modified implants 14 produced by the process 10 may include microstructures, nanofeatures, or a combination. The modified implants 14 may be characterized using electron microscopy, Raman spectroscopy, XPS, and EDS as will be described herein. Osseointegration of the modified implants 14 may be characterized by monitoring protein expression and/or mineralization corresponding to osteogenic differentiation as described herein.

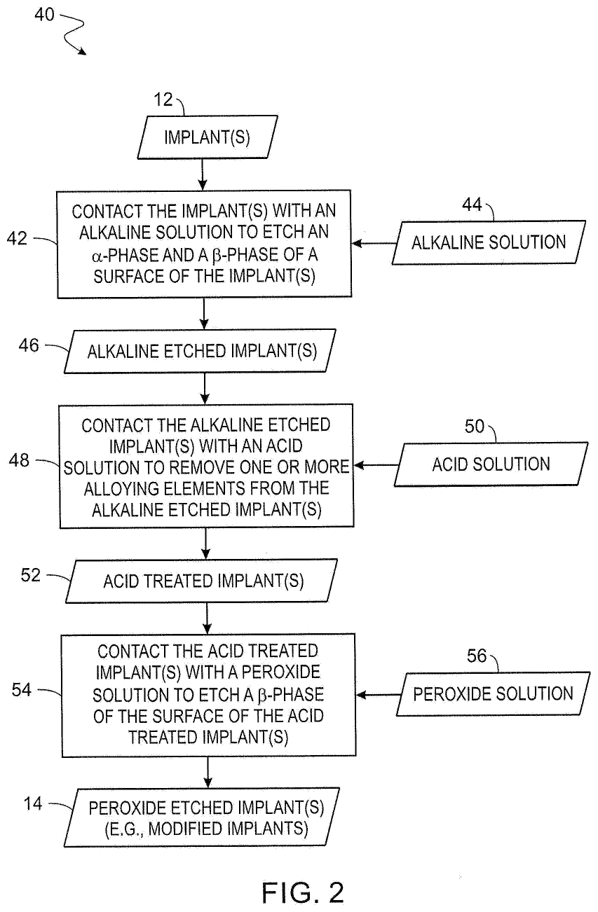

FIG. 2 is a flow diagram of a process 40 to etch an α-phase and/or a β-phase of a surface of the one or more implants 12 to produce one or more modified implants 14, in accordance with an embodiment of the present disclosure. It should be noted that certain blocks of the process 40 may be omitted and certain blocks may be repeated. At block 42 of the process 40, the implants 12 and an alkaline solution 44 are contacted to etch an α-phase and a β-phase of a surface of the implant 12. As such, block 42 may produce one or more alkaline etched implants 46. In some embodiments, the alkaline solution 44 may contain at least a base and a chelating agent. Treatment of the implants 12 with the alkaline solution 44 may generate one or more microstructures and one or more nanostructures on the surface of the alkaline etched implants 46 as described herein in reference to FIG. 4A and FIG. 4B.

At block 48 of the process 40, the alkaline etched implants 46 may be contacted with an acid solution 50 to remove one or more alloying elements from the alkaline etched implants 46. As described in FIG. 1, the acid solution 50 may include a nitric acid solution or a citric acid solution. In some embodiments, the alloying elements may include insoluble metal particulates. Block 48 of the process may produce one or more acid treated implants 52. The acid treated implants 52 may be contacted with the acid solution 50 for a short period of time (e.g., an acid flash, 1 sec to 60 secs). In this manner, the surface of the alkaline etched implants 46 may be oxidized by the acid solution 50, thereby forming one or more metal oxides on the surface of the alkaline etched implants 46.

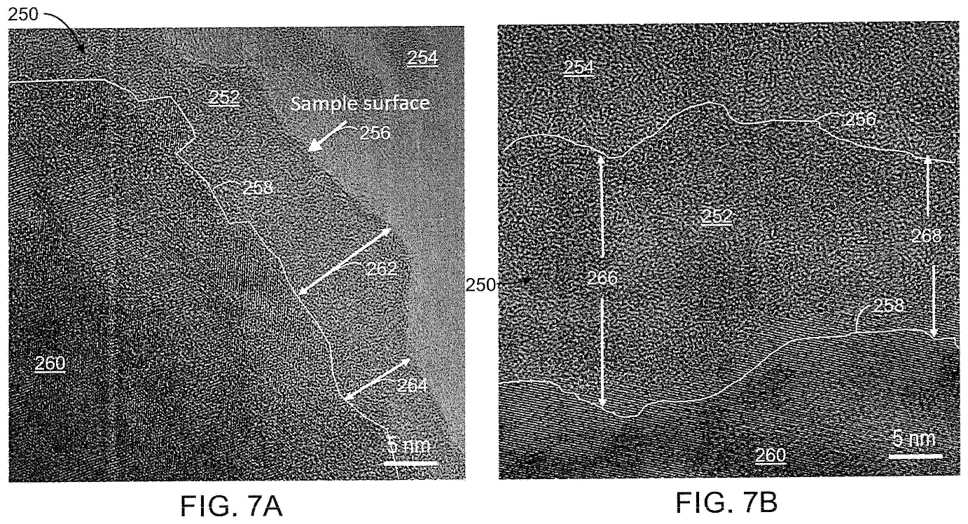

At block 54 of the process 40, the acid treated implants 52 and a peroxide solution 56 may be contacted to etch a β-phase or a combination of α-phase and β-phase of the acid treated implants 52. The peroxide solution 56 may include a hydrogen peroxide concentration between 5 to 25% by volume. Block 54 of the process may produce one or more peroxide etched implants (e.g., modified implants 14). The peroxide solution 56 may selectively etch the surface of the acid treated implants 52. Selective etching modified implants 14 may etch the β-phase of the acid treated implants 52. In an embodiment, such selective etching may be used to reduce leaching of one or more metal phases present in an implant formed from an alloy. Additionally and/or alternatively, contact of the acid treated implants 52 with the peroxide solution 56 may oxidize the surface of the acid treated implants 52. As such, the surface of the modified implants 14 may include one or more oxides. The oxides may include homogenous oxides, heterogenous oxides, or a combination thereof as described herein in reference to FIGS. 7-12. Without wishing to be bound by theory, formation of the oxides on the surface of the modified implants 14 may be influenced by selective etching of the surface of the acid treated implants 52.

### Surface Characterization of Microstructures and Nanostructures on Surfaces of Modified Implants

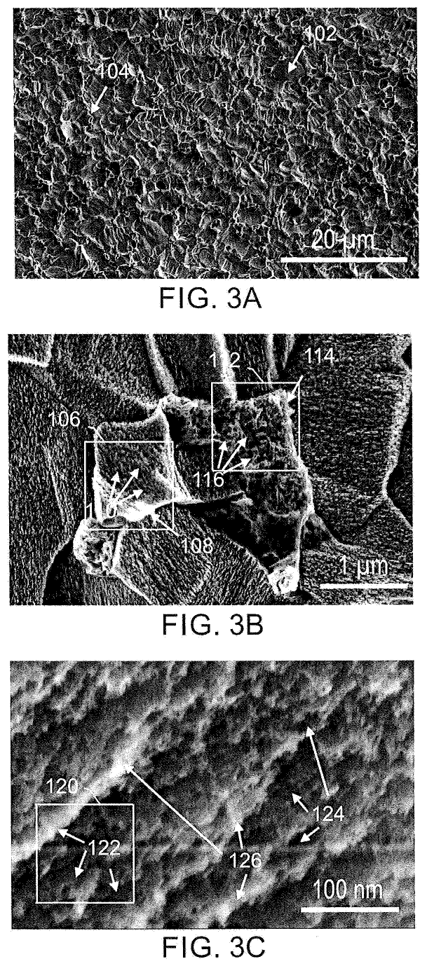

FIG. 3A is a scanning electron image (SEM) image of a portion of a modified implant 14, including a plurality of microscale structures generated by contacting an implant 12 with an alkaline solution 44 and a peroxide solution 56, in accordance with an embodiment of the present disclosure. The modified implant 14 of FIG. 3A-3C is treated according to the process 40 as described in reference FIG. 2. The implant 12 modified by process 40 to form the modified implant 14 is a wrought implant (e.g., machined implant from bar or rod stock of a metal alloy). The modified implant 14 is treated with at least the alkaline solution 44 and the peroxide solution 56 and alternatively the acid solution 50. The scale bar is 20 μm in FIG. 3A. As illustrated by arrows 102 and 104, the microscale structures generate features forming valleys and ridges, respectively, on the surface of the portion of the modified implant 14. FIG. 3B is a SEM image of a portion of the modified implant 14 of FIG. 3A, including microscale structures and nanoscale structures generated by contacting the implant 12 with the alkaline solution 44 and the peroxide solution 56, in accordance with an embodiment of the present disclosure. The scale bar is 1 μm. As illustrated by a first box 106 (e.g., 1 μm by 1 μm box) a first microscale feature 108 may include a plurality of nanoscale features 110. The plurality of nanoscale features 110 may form a corrugated surface morphology on the first microscale feature 108. Additionally, as illustrated by a second box 112 (e.g., 1 μm by 1 μm box), a second microscale feature 114 may include a plurality of nanoscale features 116. The plurality of nanoscale features 116 may include a pitted surface morphology or a corrugated texture similar to that of the alpha phase. EDS measurements of additional microscale features of which the first and second microscale feature 108, 114 are representative are rich in oxygen as measured by EDS. FIG. 3C is a SEM image of a portion of the modified implant 14 of FIG. 3A, including nanoscale structures generated by contacting the implant 12 with the alkaline solution 44 and the peroxide solution 56, in accordance with an embodiment of the present disclosure. The scale bar is 100 nm. As illustrated by a first box 120 (e.g., 100 nm by 100 nm box) a plurality of nanoscale features 122 may form a corrugated surface morphology. Additionally, as illustrated by differences in contrast within the SEM image, the portion of the modified implant 14 may include a first portion 124 of the plurality of nanoscale features 122 of a first conformation (e.g., of a particular electron density, height and/or thickness) and a second portion 126 of the nanoscales features 122 of a second conformation.

FIG. 4A is a SEM image of a portion of an alkaline etched implant 46, including a plurality of microscale structures, in accordance with an embodiment of the present disclosure. The alkaline etched implant 46 of FIGS. 4A-4C is treated according to a portion of the process 40 (e.g., block 42) as described in reference FIG. 2. The implant 12 modified by process 40 to form the alkaline etched implant 46 is a wrought implant (e.g., machined implant of a metal alloy). The surface of the alkaline etched implant 46 may be generated by contacting an implant 12 with the alkaline solution 44. It should be noted, the alkaline etched implant 46 illustrated in FIGS. 4A-4C is not treated with the peroxide solution 56. The scale bar is 1 μm in FIG. 4A. As illustrated by arrows 136 and 138, treatment of the implant 12 with the alkaline solution 44 forms microscale structures on the surface of the portion of the alkaline etched implant 46.

FIG. 4B is a SEM image, including microscale structures and nanoscale structures on a portion of a surface of an alkaline etched implant 46, in accordance with an embodiment of the present disclosure. The scale bar is 400 nm. As illustrated by arrows 140 and 142, the microscale structures generate features forming valleys and ridges on the surface of the portion of the alkaline etched implant 46. As illustrated by arrow 144, a plurality of nanoscale features may include a plurality of spikes (e.g., protrusions) generating a spiky surface morphology on the surface of the alkaline etched implant 46. FIG. 4C is a SEM image, including the plurality of nanoscale features on the portion of the surface of the alkaline etched implant 46 of FIG. 4B, in accordance with an embodiment of the present disclosure. The scale bar is 100 nm. As illustrated by arrow 146, the plurality of nanoscale features may include spikes (e.g., protrusions) on the surface of the alkaline etched implant 46. Without wishing to be bound by theory, the spikes may correspond to areas of the surface rich in α-phase character (e.g., HCP) of an alloy of the implant 12 used to generate the alkaline etched implant 46.

FIG. 5A is a SEM image of a portion of a modified implant 14 including microscale structures and nanoscale porous structures, in accordance with an embodiment of the present disclosure. The modified implant 14 of FIG. 5A-5C is treated according to the process 40 as described in reference to FIG. 2. The implant 12 modified by process 40 to form the modified implant 14 is a wrought implant (e.g., machined implant of a metal alloy). The peroxide solution 56 used during surface modification of the modified implant 14 of FIG. 5A-5C included about 20% by volume hydrogen peroxide. Without wishing to be bound by theory, treatment of the implant 12 with a higher concentration of hydrogen peroxide solution such as 20% hydrogen peroxide may form nanoscale porous structures on the α-phase, generating a porous surface morphology of the surface of the modified implant 14 not present following treatment with the alkaline solution 44. The scale bar of FIG. 5A is 1 μm. As illustrated by a first box 180 (e.g., 1 μm by 1 μm box) a first microscale feature 182 may include a plurality of nanoscale features 184. The plurality of nanoscale features 184 may form a corrugated surface morphology on the first microscale feature 182. Additionally, as illustrated by a second box 186 (e.g., 1 μm by 1 μm box), a second microscale feature 188 may include a plurality of nanoscale features 190. FIG. 5B is a SEM image of a portion of the modified implant 14 of FIG. 5A increased in magnification, including microscale structures and nanoscale porous structures, in accordance with an embodiment of the present disclosure. The scale bar is 400 nm. As illustrated by a first box 192 (e.g., 400 nm by 400 nm box) a plurality of nanoscale porous features 194 may form a corrugated surface morphology with one or more pores. FIG. 5C is a SEM image of a portion of the modified implant 14 of FIG. 5B, including nanoscale porous structures, in accordance with an embodiment of the present disclosure. The scale bar is 100 nm. As illustrated by arrow 196, a plurality of nanoscale porous features may include nanopores (e.g., nanoholes) on the surface of the modified implant 14 treated with the 20% hydrogen peroxide solution.

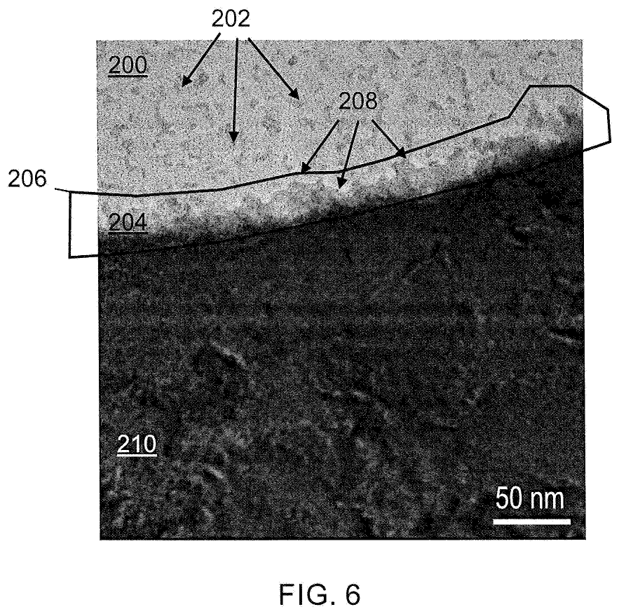

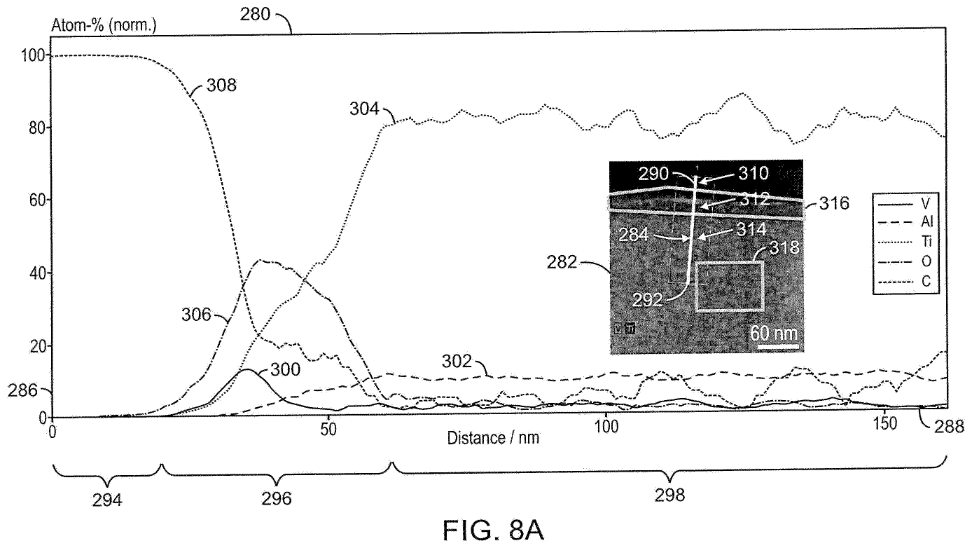

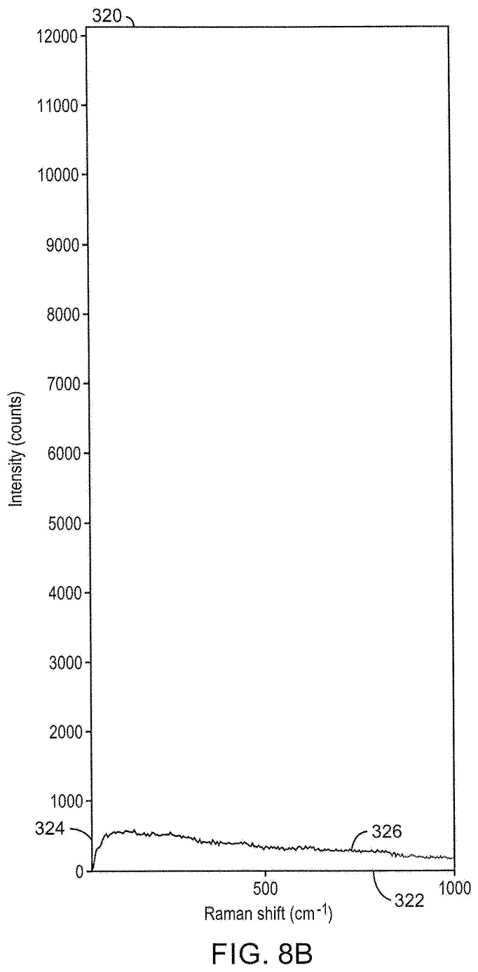

FIG. 6 is transmission electron microscopy (TEM) image from the cross section following focused ion beam milling (FIB) of a modified implant 14 (e.g., a sample disc representative of an implantable device) including oxide formation, in accordance with an embodiment of the present disclosure. The modified implant 14 of FIG. 6 is treated according to the process 40 as described in reference FIG. 2. That is, the modified implant 14 is treated with at least the alkaline solution 44 and the peroxide solution 56 and additionally the acid solution 50. The implant 12 modified by process 40 to form the modified implant 14 is a wrought implant (e.g., machined implant of a metal alloy). The scale bar is 50 nm. A cross section of the modified implant 14 is milled using FIB and is imaged on a carbon protective layer 200 as shown by arrows 202. The carbon protective layer 200 is a sacrificial layer to prevent damage to the surface of the modified implant 14 during sample preparation (e.g., FIB milling). The modified implant 14 includes a surface oxide 204 as illustrated by box 206. The surface oxide 204 may include a heterogenous surface morphology as illustrated by arrows 208. A thickness of the surface oxide 204 may range between approximately 2 nm and 50 nm. As will be described in reference to FIGS. 8A-11B, the surface oxide 204 may include homogenous oxides, heterogenous oxides, or a combination thereof. The modified implant 14 includes a bulk portion 210 of higher electron density. The bulk portion 210 of the modified implant 14 may include metals, metal alloys, or a combination thereof.

FIG. 7A is a first embodiment of a high resolution TEM (HR-TEM) image of a modified implant 14 showing an amorphous lattice 250 of the modified implant 14 and formation of a surface oxide, in accordance with an embodiment of the present disclosure. FIG. 7B is a second embodiment of a high resolution TEM (HR-TEM) image of a modified implant 14 showing an amorphous lattice 250 of the modified implant 14 and formation of a surface oxide, in accordance with an embodiment of the present disclosure. The modified implant 14 of FIGS. 7A and 7B are treated according to the process 40 as described in reference FIG. 2. That is, the modified implant 14 is treated with at least the alkaline solution 44 and the peroxide solution 56 and additionally the acid solution 50. The implant 12 modified by process 40 to form the modified implant 14 is a wrought implant (e.g., machined implant of a metal alloy). The scale bars of FIGS. 7A and 7B are 5 nm. As shown, a cross section of the modified implant 14 is imaged along with the carbon protective layer 254. Arrows 256 illustrates a surface sample of the surface oxide 252 of the modified implant 14. Lines 258 illustrate a grain boundary between the surface oxide 252 and a bulk portion 260 of the modified implant 14. A thickness of the surface oxide 252 may vary across the modified implant 14 (e.g., a thickness between the surface sample and the grain boundary) as illustrated by arrows 262 and 264 of FIG. 7A and arrows 266 and 268 of FIG. 7B. A thickness of the surface oxide 252 may range between approximately 2 nm and 50 nm. The bulk portion 260 may include crystal lattice structures corresponding to an α-phase and/or a β-phase having HCP character and BCC character, respectively.

### Oxide Characterization of Surfaces of Modified Implants

FIG. 8A-FIG. 11B include electron dispersive spectroscopy (EDS), TEM, and Raman characterization of surfaces of one or more modified implants 14. The modified implants 14 analyzed in FIG. 8A-11B are treated according to the process 40 as described in reference FIG. 2. That is, the modified implant 14 is treated with at least the alkaline solution 44 and the peroxide solution 56 and alternatively the acid solution 50. The implant 12 modified by process 40 to form the modified implant 14 is a wrought implant (e.g., machined implant of a metal alloy).

FIG. 8A is an EDS graph 280 of a portion of the modified implant 14 illustrating formation of vanadium oxide, including an inset TEM with EDS map of the image 282 of a correlated measurement area of the modified implant 14, in accordance with an embodiment of the present disclosure. The portion of the modified implant 14 is selected from a portion of a microstructure forming a valley within the surface morphology of the modified implant 14 as illustrated in FIG. 3A and FIG. 3B. The depth of the EDS graph 280 into modified implant is approximately 140 nm. The field of view (FOV) for the inset TEM with EDS map image 282 is 300 nm. The scale bar of the TEM image 282 is 60 nm and an EDS map is overlayed on the TEM image 282. The EDS graph 280 provides elemental concentration along a selected line 284 of the TEM image 282. A y-axis 286 corresponds to an elemental composition percentage. An x-axis 288 illustrates a distance in nanometers of the selected line 284 of the TEM image 282, where zero corresponds to a first end 290 of the selected line 284 and 160 corresponds to second end 292 of the selected line 284. As shown, the selected line 284 of the TEM image 282 measured by the EDS graph 280 passed through a background region 294, a surface oxide region 296, and a bulk region 298. The elemental composition of various elements is detected in varying amounts across the selected line 284. The elemental compositions of various elements are represented based on elemental abundance of vanadium, aluminum, titanium, oxygen, and carbon. As such, the EDS graph 280 includes a vanadium profile 300, an aluminum profile 302, a titanium profile 304, an oxygen profile 306, and a carbon profile 308.

In the background region 294 (e.g., zero nm to 20 nm) the elemental composition is formed mostly by carbon, shown by the carbon profile 308. The background region 294 corresponds to measuring the carbon protective layer 310 used to protect the modified implant 14 during FIB milling. As the EDS line scan of the selected line 284 approaches the surface oxide region 296 (e.g., 20 nm to 60 nm) corresponding to surface oxide 312 illustrated in the TEM image 282, the elemental composition of the vanadium profile 300, the titanium profile 304, the aluminum profile 302, and the oxygen profile 306 increase as the carbon profile 308 decreases. The EDS line scan from 30 nm to 40 nm illustrates the presence of vanadium oxide at the surface oxide 312 (e.g., outer surface oxide) of the modified implant 14. The EDS line scan from 40 nm to 60 nm illustrates the presence of aluminum oxide near the surface oxide 312 of the modified implant 14. The EDS line scan from 30 nm to 60 nm illustrates the presence of titanium oxide. As the EDS line scan of the selected line 284 approaches the bulk region 298 (e.g., 60 nm to 160 nm) corresponding to bulk portions 314 of the modified implant 14 illustrated in the TEM image 282, the elemental composition of the vanadium profile 300 and the oxygen profile 306 decrease as the titanium profile 304 and the aluminum profile 302 remains consistent.

Additional elemental analysis of the surface oxide 312 and the bulk portions 314 of the modified implant 14 are shown in Table 1 and Table 2, respectively. Table 1 shows the elemental composition of a first box 316 of the TEM image 282 with the EDS overlay. Table 2 shows the elemental composition of a second box 318 of the TEM image 282. Comparison of Table 1 and Table 2 shows an increased amount of vanadium and oxygen in the surface oxide 312 as compared to the bulk 314. Without wishing to be bound by theory, the increased amounts of vanadium and oxygen at the surface of the modified implant 14 is indicative of the presence of vanadium oxide.

TABLE 1
Elemental composition corresponding to the
surface oxide of the modified implant

| Element | Net | Mass C. [wt. %] | Atom C. [wt. %] |
|---|---|---|---|
| V | 3296 | 12.62 | 7.94 |
| Al | 1244 | 4.13 | 4.91 |
| Ti | 16153 | 59.64 | 39.90 |
| O | 5730 | 23.60 | 47.26 |

TABLE 2
Elemental composition corresponding
to the bulk of the modified implant

| Element | Net | Mass C. [wt. %] | Atom C. [wt. %] |
|---|---|---|---|
| V | 1733 | 2.52 | 2.23 |
| Al | 4951 | 6.25 | 10.45 |
| Ti | 64590 | 90.57 | 85.44 |
| O | 424 | 0.66 | 1.87 |

FIG. 8B is Raman graph 320 of the portion of the modified implant 14 of FIG. 8A, in accordance with an embodiment of the present disclosure. It should be noted, the Raman graph 320 includes the FOV of the TEM image 282 (e.g., 300 nm) as the spot size of the Raman measurement ranges from about 500 nm to 700 nm. The Raman graph 320 includes a x-axis 322 of a Raman shift (cm⁻¹) and a y-axis 324 of an intensity (counts). As shown by line 326, the Raman signal is weak for the area of the TEM image.

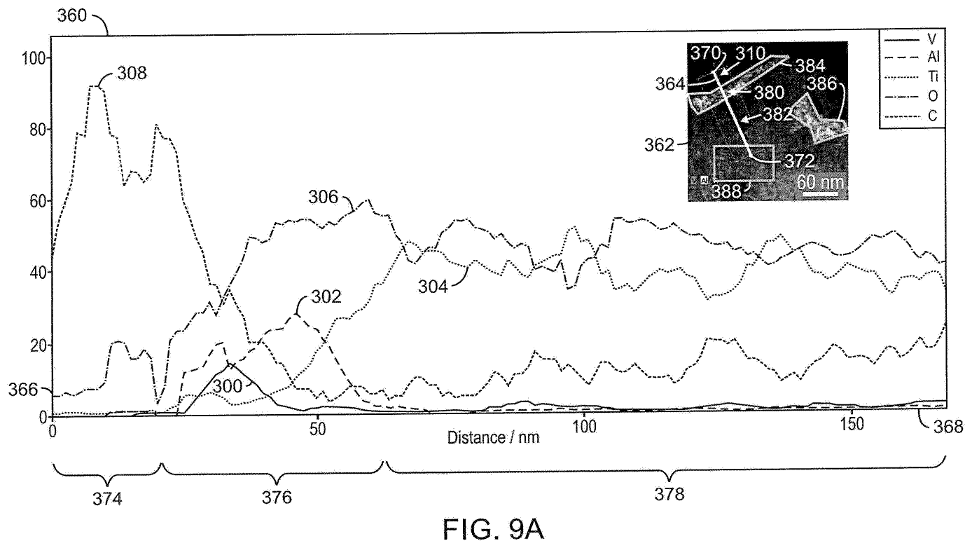

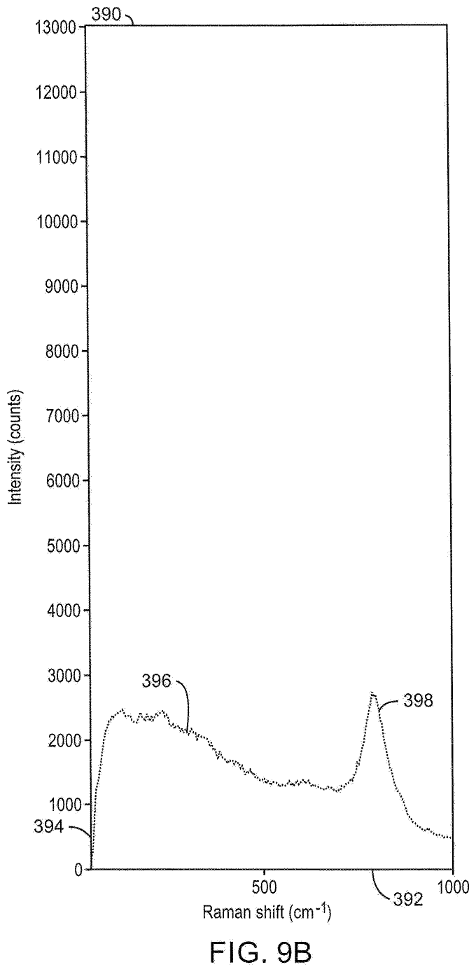

FIG. 9A is an EDS graph 360 of a portion of the modified implant 14 illustrating formation of aluminum oxide, including an inset TEM with EDS map of the image 362 of a correlated measurement area of the modified implant 14, in accordance with an embodiment of the present disclosure. The portion of the modified implant 14 is selected from a portion of a microstructure forming a raised structure within the surface morphology of the modified implant 14 as illustrated in FIG. 3A and FIG. 3B. The depth of the EDS graph 360 into the modified implant is approximately 140 nm. The field of view (FOV) for the inset TEM with EDS map image 362 is 300 nm. The scale bar of the TEM image 362 is 60 nm and an EDS map is overlayed on the TEM image 362. The EDS graph 360 provides elemental concentration along a selected line 364 of the TEM image 362. A y-axis 366 corresponds to an elemental composition percentage. An x-axis 368 illustrates a distance in nanometers of the selected line 364 of the TEM image 362, where zero corresponds to a first end 370 of the selected line 364 and 170 corresponds to second end 372 of the selected line 364. As shown, the selected line 364 of the TEM image 362 measured by the EDS graph 360 passed through a background region 374, a surface oxide region 376, and a bulk region 378. The elemental composition of various elements is detected in varying amounts across the selected line 364. The elemental compositions of various elements are represented based on elemental abundance of vanadium, aluminum, titanium, oxygen, and carbon. As such, the EDS graph 360 includes the vanadium profile 300, the aluminum profile 302, the titanium profile 304, the oxygen profile 306, and the carbon profile 308.

In the background region 374 (e.g., zero nm to 20 nm) the elemental composition is formed mostly by carbon, shown by the carbon profile 308. The background region 374 corresponds to measuring the carbon protective layer 310 used to protect the modified implant 14 during FIB milling. As the EDS line scan of the selected line 364 approaches the surface oxide region 376 (e.g., 30 nm to 60 nm) corresponding to surface oxide 380 illustrated in the TEM image 362, the elemental composition of the vanadium profile 300, the aluminum profile 302, the titanium profile 304 and the oxygen profile 306 increase as the carbon profile 308 decreases. The EDS line scan from 35 nm to 55 nm illustrates the presence of aluminum oxide at the surface oxide 380 of the modified implant 14. As the EDS line scan of the selected line 364 approaches the bulk region 378 (e.g., 60 nm to 160 nm) corresponding to bulk portions 382 of the modified implant 14 illustrated in the TEM image 362, the elemental composition of the vanadium profile 300 and the aluminum profile 302 decrease as the oxygen profile 306 remains consistent and the titanium profile 304 increases.

Additional elemental analysis of a first portion 384 the surface oxide 380, a second portion 386 of the surface oxide 380, and the bulk portions 382 of the modified implant 14 are shown in Table 3, Table 4, and Table 5, respectively. Table 3 shows the elemental composition of the first portion 384 of the TEM image 362 with the EDS overlay. Table 4 shows the elemental composition of the second portion 386 of the TEM image 362 with the EDS overlay. Table 5 shows the elemental composition of a box 388 of the bulk 382 of the TEM image 362. Comparison of Tables 3 and 4 to Table 5 shows an increased amount of aluminum in the surface oxide 380 as compared to the bulk 382. Without wishing to be bound by theory, the increased amounts of aluminum at the surface of the modified implant 14 are indicative of the presence of aluminum oxide.

TABLE 3
Elemental composition corresponding to the first portion
of the surface oxide of the modified implant of FIG. 9A

| Element | Net | Mass C. [wt. %] | Atom C. [wt. %] |
|---|---|---|---|
| V | 275 | 9.15 | 4.20 |
| Al | 953 | 27.57 | 23.88 |
| Ti | 657 | 21.11 | 10.31 |
| O | 1175 | 42.17 | 61.61 |

TABLE 4
Elemental composition corresponding to the second portion
of the surface oxide of the modified implant of FIG. 9A

| Element | Net | Mass C. [wt. %] | Atom C. [wt. %] |
|---|---|---|---|
| V | 234 | 5.25 | 2.49 |
| Al | 1383 | 26.85 | 23.98 |
| Ti | 1329 | 28.67 | 14.43 |
| O | 1630 | 39.23 | 59.10 |

TABLE 5
Elemental composition corresponding to the
bulk of the modified implant of FIG. 9A

| Element | Net | Mass C. [wt. %] | Atom C. [wt. %] |
|---|---|---|---|
| V | 342 | 1.80 | 1.09 |
| Al | 61 | 0.28 | 0.32 |
| Ti | 13843 | 70.12 | 45.09 |
| O | 4921 | 27.81 | 53.51 |

FIG. 9B is Raman graph 390 of the portion of the modified implant 14 of FIG. 9A, in accordance with an embodiment of the present disclosure. It should be noted, the Raman graph 390 includes the FOV of the TEM image 362 (e.g., 300 nm) as the spot size of the Raman measurement ranges from about 500 nm to 700 nm. The Raman graph 390 includes an x-axis 392 of a Raman shift (cm⁻¹) and a y-axis 394 of an intensity (counts). As shown by line 396, the Raman signal has a peak 398 (e.g., approximately 800 cm⁻¹) indicative of presence of aluminum oxide.

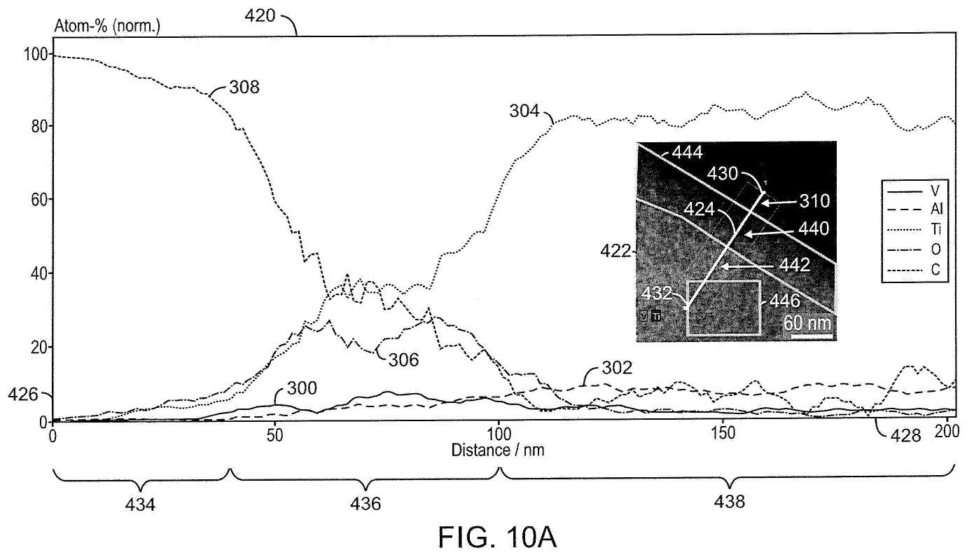

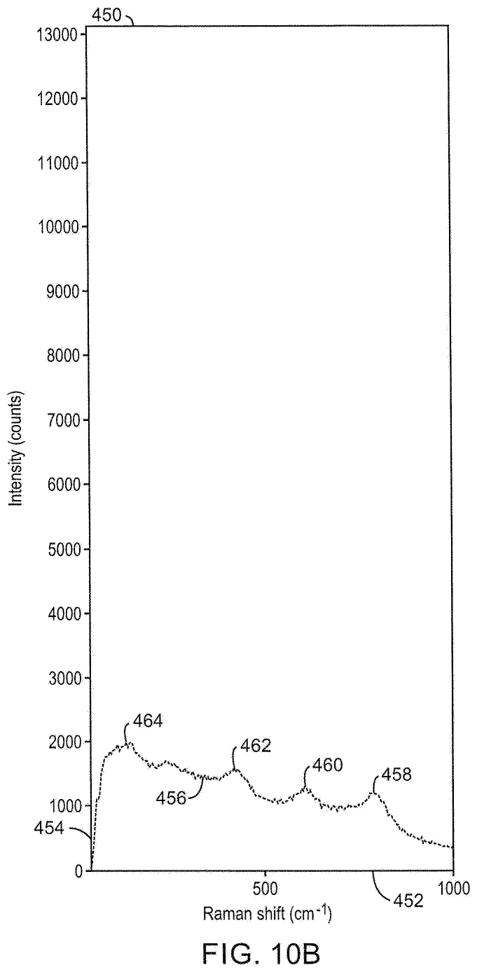

FIG. 10A is an EDS graph 420 of a portion of the modified implant 14 illustrating formation of aluminum oxide and vanadium oxide, including an inset TEM with EDS map of image 422 of a correlated measurement area of the modified implant 14, in accordance with an embodiment of the present disclosure. The portion of the modified implant 14 is selected from a portion of a microstructure forming a ridge within the surface morphology of the modified implant 14 as illustrated in FIG. 3A and FIG. 3B. The depth of the EDS graph 420 into the modified implant is approximately 160 nm. The field of view (FOV) for the inset TEM with EDS map image 422 is 300 nm. The scale bar of the TEM image 422 is 60 nm and an EDS map is overlayed on the TEM image 422. The EDS graph 420 provides elemental concentration along a selected line 424 of the TEM image 422. A y-axis 426 corresponds to an elemental composition percentage. An x-axis 428 illustrates a distance in nanometers of the selected line 424 of the TEM image 422, where zero corresponds to a first end 430 of the selected line 424 corresponds to second end 432 of the selected line 424. As shown, the selected line 424 of the TEM image 422 measured by the EDS graph 420 passed through a background region 434, a surface oxide region 436, and a bulk region 438. The elemental composition of various elements is detected in varying amounts across the selected line 424. The elemental compositions of various elements are represented based on elemental abundance of vanadium, aluminum, titanium, oxygen, and carbon. As such, the EDS graph 420 includes the vanadium profile 300, the aluminum profile 302, the titanium profile 304, the oxygen profile 306, and the carbon profile 308.

In the background region 434 (e.g., zero nm to 40 nm) the elemental composition is formed mostly by carbon, shown by the carbon profile 308. The background region 434 corresponds to measuring the carbon protective layer 310 used to protect the modified implant 14 during FIB. As the EDS line scan of the selected line 424 approaches the surface oxide region 436 (e.g., 40 nm to 100 nm) corresponding to surface oxide 440 illustrated in the TEM image 422, the elemental composition of the vanadium profile 300, the aluminum profile 302, the titanium profile 304, and the oxygen profile 306 increase as the carbon profile 308 decreases. The EDS line scan (e.g., from 50 nm to 100 nm) illustrates the presence of vanadium oxide, titanium oxide, and aluminum oxide at the surface oxide 440 of the modified implant 14. As the EDS line scan of the selected line 424 approaches the bulk region 438 (e.g., 100 nm to 200 nm) corresponding to bulk 442 of the modified implant 14 illustrated in the TEM image 422, the elemental composition of the vanadium profile 300, and the oxygen profile 306 decrease and the aluminum profile 302 remains constant as the titanium profile 304 increases.

Additional elemental analysis of the surface oxide 440 and the bulk portions 442 of the modified implant 14 are shown in Table 6 and Table 7, respectively. Table 6 shows the elemental composition of a first box 444 of the TEM image 422 with the EDS overlay. Table 7 shows the elemental composition of a second box 446 of the TEM image 422. Comparison of Table 6 and Table 7 shows an increased amount of vanadium and oxygen in the surface oxide 440 as compared to the bulk 442. Without wishing to be bound by theory, the increased amounts of vanadium and oxygen at the surface of the modified implant 14 is indicative of the presence of vanadium oxide.

TABLE 6
Elemental composition corresponding to the surface
oxide of the modified implant of FIG. 10A

| Element | Net | Mass C. [wt. %] | Atom C. [wt. %] |
|---|---|---|---|
| V | 6947 | 10.31 | 7.44 |
| Al | 3287 | 4.23 | 5.77 |
| Ti | 50052 | 71.63 | 55.01 |
| O | 8662 | 13.83 | 31.78 |

TABLE 7
Elemental composition corresponding to the
bulk of the modified implant of FIG. 10A

| Element | Net | Mass C. [wt. %] | Atom C. [wt. %] |
|---|---|---|---|
| V | 1253 | 2.52 | 2.27 |
| Al | 2884 | 5.04 | 8.54 |
| Ti | 47328 | 91.97 | 87.87 |
| O | 213 | 0.46 | 1.32 |

FIG. 10B is Raman graph 450 of the portion of the modified implant 14 of FIG. 10A, in accordance with an embodiment of the present disclosure. It should be noted, the Raman graph 450 includes the FOV of the TEM image 422 (e.g., 300 nm) as the spot size of the Raman measurement ranges from about 500 nm to 700 nm. The Raman graph 450 includes an x-axis 452 of a Raman shift (cm⁻¹) and a y-axis 454 of an intensity (counts). As shown by line 456, the Raman signal has a first peak 458 (e.g., approximately 800 cm⁻¹) indicative of presence of aluminum oxide, a second peak 460 (e.g., approximately 600 cm⁻¹) and a third peak 462 (e.g., approximately 400 cm⁻¹) indicative of vanadium oxide, and a fourth peak 464 (e.g., approximately 150 cm⁻¹) indicative of titanium oxide.

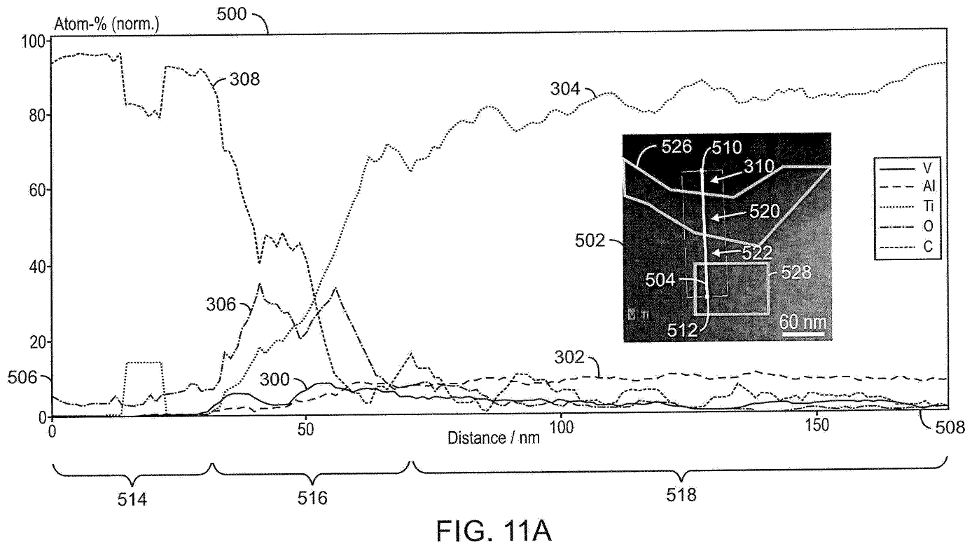

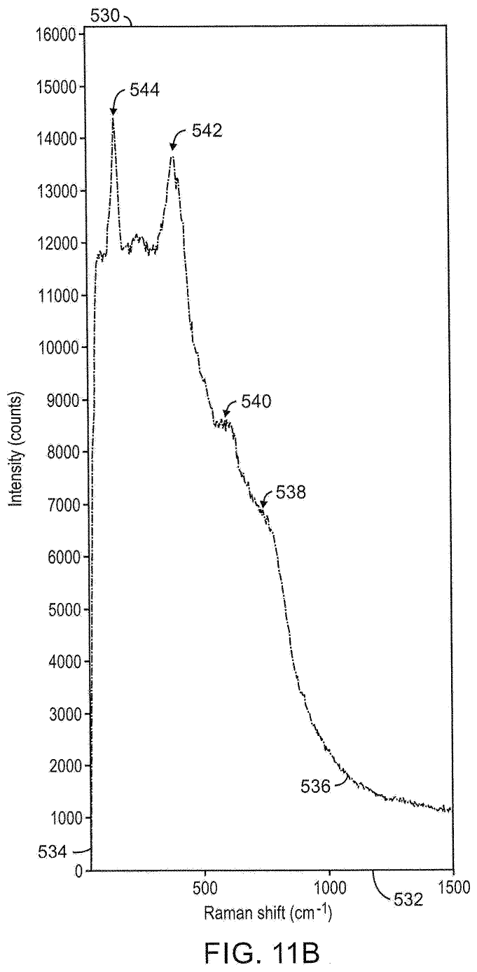

FIG. 11A is an EDS graph 500 of a portion of the modified implant 14 illustrating formation of titanium oxide and vanadium oxide, including an inset TEM with an EDS map image 502 of a correlated measurement area of the modified implant 14, in accordance with an embodiment of the present disclosure. The portion of the modified implant 14 is selected from a portion of a microstructure forming a valley within the surface morphology of the modified implant 14 as illustrated in FIG. 3A and FIG. 3B. The depth of the EDS graph 500 into the modified implant is approximately 145 nm. The field of view (FOV) for the inset TEM with EDS map image 502 is 300 nm. The scale bar of the TEM image 502 is 60 nm and an EDS map is overlayed on the TEM image 502. The EDS graph 500 provides elemental concentration along a selected line 504 of the TEM image 502. A y-axis 506 corresponds to an elemental composition percentage. An x-axis 508 illustrates a distance in nanometers of the selected line 504 of the TEM image 502, where zero corresponds to a first end 510 of the selected line 504 and 175 nm corresponds to second end 512 of the selected line 504. As shown, the selected line 504 of the TEM image 502 measured by the EDS graph 500 passed through a background region 514, a surface oxide region 516, and a bulk region 518. The elemental composition of various elements is detected in varying amounts across the selected line 504. The elemental compositions of various elements are represented based on elemental abundance of vanadium, aluminum, titanium, oxygen, and carbon. As such, the EDS graph 500 includes the vanadium profile 300, the aluminum profile 302, the titanium profile 304, the oxygen profile 306, and the carbon profile 308.

In the background region 514 (e.g., 0 nm to 30 nm) the elemental composition is formed mostly by carbon, shown by the carbon profile 308. The background region 514 corresponds to measuring the carbon protective layer 310 used to protect the modified implant 14 during FIB milling. As the EDS line scan of the selected line 504 approaches the surface oxide region 516 (e.g., 30 nm to 70 nm) corresponding to surface oxide 520 illustrated in the TEM image 502, the elemental composition of the vanadium profile 300, the titanium profile 304, the aluminum profile 302, and the oxygen profile 306 increase as the carbon profile 308 decreases. The EDS line scan from 30 nm to 60 nm illustrates the presence of vanadium oxide, aluminum oxide, and titanium oxide at the surface oxide 520 of the modified implant 14. As the EDS line scan of the selected line 504 approaches the bulk region 518 (e.g., 70 nm to 175 nm) corresponding to bulk 522 of the modified implant 14 illustrated in the TEM image 502, the elemental composition of the vanadium profile 300 and the oxygen profile 306 decrease and the aluminum profile 302 remains constant as the titanium profile 304 continues to increase.

Additional elemental analysis of the surface oxide 520 and the bulk portions 522 of the modified implant 14 are shown in Table 8 and Table 9, respectively. Table 8 shows the elemental composition of a first box 524 of the TEM image 502 with the EDS overlay. Table 9 shows the elemental composition of a second box 526 of the TEM image 422. Comparison of Table 8 and Table 9 shows an increased amount of vanadium and oxygen in the surface oxide 520 as compared to the bulk 522. Without wishing to be bound by theory, the increased amounts of vanadium and oxygen at the surface of the modified implant 14 are indicative of the presence of vanadium oxide.

TABLE 8
Elemental composition corresponding to the surface
oxide of the modified implant of FIG. 11A

| Element | Net | Mass C. [wt. %] | Atom C. [wt. %] |
|---|---|---|---|
| V | 2570 | 10.93 | 7.71 |
| Al | 1163 | 4.29 | 5.71 |
| Ti | 16941 | 69.47 | 52.17 |
| O | 3347 | 15.31 | 34.41 |

TABLE 9
Elemental composition corresponding to the
bulk of the modified implant of FIG. 11A

| Element | Net | Mass C. [wt. %] | Atom C. [wt. %] |
|---|---|---|---|
| V | 1855 | 2.60 | 2.35 |
| Al | 4312 | 5.24 | 8.93 |
| Ti | 68104 | 92.04 | 88.37 |
| O | 81 | 0.12 | 0.35 |

FIG. 11B is Raman graph 530 of the portion of the modified implant 14 of FIG. 10A, in accordance with an embodiment of the present disclosure. It should be noted, the Raman graph 530 includes the FOV of the TEM image 502 (e.g., 300 nm) as the spot size of the Raman measurement ranges from about 500 nm to 700 nm. The Raman graph 530 includes an x-axis 532 of a Raman shift (cm⁻¹) and a y-axis 534 of an intensity (counts). As shown by line 536, the Raman signal has a first peak 538 (e.g., approximately 800 cm⁻¹) indicative of presence of aluminum oxide, a second peak 540 (e.g., approximately 600 cm⁻¹) and a third peak 542 (e.g., approximately 400 cm⁻¹) indicative of vanadium oxide, and a fourth peak 544 (e.g., approximately 150 cm⁻¹) indicative of titanium oxide.

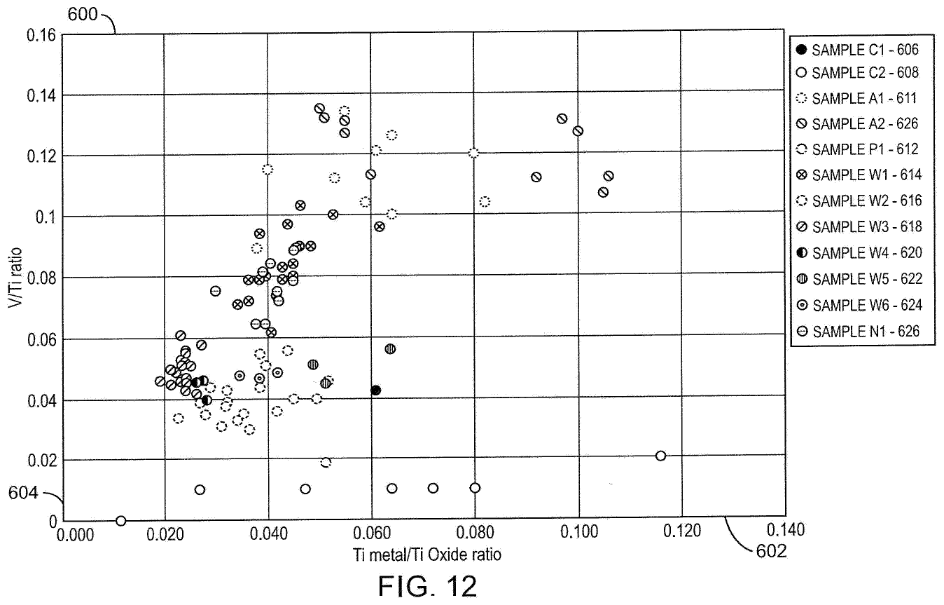

FIG. 12 is a graph 600 of metal to oxide ratios of implants and treated implants measured using X-ray photoelectron spectroscopy (XPS), in accordance with an embodiment of the present disclosure. The graph 600 includes an x-axis 602 of a titanium metal to titanium oxide ratio and a y-axis 604 of a vanadium metal to titanium metal ratio. The graph 600 includes a variety of datapoints and datasets for a variety of implants with varied reaction conditions. The graph 600 includes datasets corresponding to comparative samples, a sample C1 606, a sample C2 608, a sample A1 610, a sample A2 611, and a sample P1 612. The graph 600 also includes datasets corresponding to implants 12 treated following the process 40 including wrought modified implants corresponding to a sample W1 614, a sample W2 616, a sample W3 618, a sample W4 620, a sample W5 622, a sample W6 624 and an additively manufactured implant corresponding to a sample N1 626.

Sample C1 606 corresponds to an untreated implant (e.g., representative disc of a machined implant with no surface modification). Sample C2 608 corresponds to a comparative sample in which an implant 12 is modified using a line-of-sight surface modification technique (e.g., grit-blasting and acid etching techniques). Sample A1 610 corresponds to an alkaline etched implant 46 (e.g., representative disc of a wrought alkaline etched implant) in which a surface of an implant 12 is treated only with the alkaline solution 44. Sample A1 611 correspond to an additively manufactured implant treated with the alkaline solution 44 (e.g., an additively manufacture alkaline etched implant). Sample P1 612 corresponds to an implant treated only with the peroxide solution 56. Samples W1-W6 include wrought implants (e.g., machined implant of a metal alloy) treated using the alkaline solution 44, the acid solution 50, and the peroxide solution 56 as described in reference to FIG. 2. Sample N1 includes an additively manufacture implant (e.g., representative disc of a the additively manufacture implant) treated using the alkaline solution 44, the acid solution 50, and the peroxide solution 56 as described in reference to FIG. 2. Table 10 shows reaction conditions of Samples W1-W6 and Sample N1 shown in FIG. 12. The NaOH concentration may range from about 20% to about 30% of the alkaline solution. The chelating concentration may range from about 3% to about 6% of the alkaline solution. The temperature of the alkaline solution may range from 85 °C to less than 100 °C. The etch time of Samples W1-W6 and Sample N1 in the alkaline solution may range from about 18 mins to about 30 mins. The H₂O₂ concentration may range from about 5% to about 11% of the peroxide solution. The temperature of the peroxide solution may range from about 40 °C to about 60 °C. The time the Samples W1-W6 and Sample N1 are treated in the H₂O₂ solution may range from about 10 mins to about 20 mins.

TABLE 10
Reaction conditions of the modified implants of FIG. 12

| | W1 | W2 | W3 | W4 | W5 | W6 | N1 |
|---|---|---|---|---|---|---|---|
| Alkaline | | | | | | | |
| Solution | | | | | | | |
| NaOH | 24.5-25.5 | 21.5-22.5 | 24.5-25.5 | 20-26 | 20-26 | 20-26 | 20-26 |
| concentration | | | | | | | |
| (%) | | | | | | | |
| Chelating agent | 5.5-6 | 3-3.5 | 5.5-6 | 3-6 | 3-6 | 3-6 | 3-6 |
| concentration | | | | | | | |
| (%) | | | | | | | |
| Temperature | 92-98 | 85-88 | 92-98 | 85-98 | 85-98 | 85-98 | 85-98 |
| (°C) | | | | | | | |
| Etch Time (min) | 24.5-25 | 20-20.5 | 24.5-25 | 18-30 | 18-30 | 18-30 | 18-30 |
| Peroxide | | | | | | | |
| Solution | | | | | | | |
| H₂0₂ | 5.5-6 | 10-10.5 | 7.5-8 | 5.5-10.5 | 5.5-10.5 | 5.5-10.5 | 5.5-10.5 |
| concentration | | | | | | | |
| (%) | | | | | | | |
| Temperature | 43-55 | 43-55 | 43-55 | 43-55 | 43-55 | 43-55 | 43-55 |
| (°C) | | | | | | | |
| Time (mins) | 13.5 | 13.5 | 13.5 | 10-20 | 10-20 | 10-20 | 10-20 |

Table 11 includes average oxide and metal to oxide ratios of the samples shown in FIG. 12. As shown in FIG. 12 and Table 11, sample A1 610 has a higher amount of vanadium when compared to Samples W1-W6 (e.g., 614, 616, 618, 620, 622, 624). An average vanadium metal to titanium metal of Sample W1 614 is 0.08. An average vanadium metal to titanium metal of sample A1 610 is 0.11. The average vanadium metal to titanium metal of Sample W2 618 is 0.05 and the average vanadium metal to titanium metal for the of Sample W3 616 is 0.04. Without wishing to be bound by theory, treatment of alkaline etched implants 46 with the peroxide solution 56 may reduce an amount of vanadium on the surface of the implants. Vanadium may be present in increased concentrations in the β-phase of implants after treatment with the alkaline solution 44. As such, treatment with the peroxide solution 56 may selectively etch the β-phase of implants. Comparison of Sample W1 614, Sample W2 616, and Sample W3 618 demonstrate that increasing concentration of hydrogen peroxide in the peroxide solution 56 may correspond to increased reduction in an amount of vanadium present in respective implants. Samples W1-W6 (e.g., 614, 616, 618, 620, 622, 624) have a vanadium to titanium ratio greater than 0.02. In some embodiments, the vanadium metal to titanium metal ratios of the modified implants may range from about 0.02 to about 0.11, about 0.04 to about 0.11, about 0.02 to about 0.08, about 0.02 to about 0.11, greater than 0.04, greater than 0.06, and the like. The titanium dioxide to titanium metal ratios of the modified implants may range from about 19 to about 37, about 20 to about 29, about 20.2 to about 36.9, about 20.2 to about 26.5, greater than 19, greater than 20, greater than 25, and the like. The titanium metal to titanium dioxide ratios of the modified implants may range from about 0.02 to about 0.06, about 0.02 to about 0.05, about 0.02 to about 0.04, less than 0.05, less than or equal to 0.04, and the like.

TABLE 11
Averaged metal and oxide surface characteristics
of the implants of FIG. 12

| | TiO₂/Ti | Ti/TiO₂ | V/Ti |
|---|---|---|---|
| C1 | 16.5 | 0.06 | 0.04 |
| C2 | 28.5 | 0.06 | 0.01 |
| A1 | 19.1 | 0.06 | 0.11 |
| P1 | 20.0 | 0.05 | 0.02 |
| W1 | 23.5 | 0.04 | 0.08 |
| W2 | 29.0 | 0.04 | 0.04 |
| W3 | 20.2 | 0.02 | 0.05 |
| W4 | 36.9 | 0.03 | 0.04 |
| W5 | 26.1 | 0.04 | 0.06 |
| W6 | 26.5 | 0.04 | 0.05 |

As shown in FIG. 12 and Table 11, Sample A1 610 has a higher titanium metal to titanium dioxide ratio when compared to Samples W1-W6 (e.g., 614, 616, 618, 620, 622, 624). Without wishing to be bound by theory, treatment of alkaline etched implants 46 with the peroxide solution 56 may increase an amount of oxide formation on the surface of implants. Samples W1-W6 (e.g., 614, 616, 618, 620, 622, 624) have a titanium oxide to titanium metal ratio greater than 20 parts titanium to 1 part titanium dioxide.

Table 12 includes atomic force microscopy (AFM) measurements with a capture area of 500 nm×500 nm for the unmodified implant and the first comparative sample (e.g., line-of-sight modified implant). The root mean square (e.g., Sq) and the maximum peak-to-valley distance (e.g., Sz) and/or a mean height (e.g., Sa) of the modified implant 14 and the first comparative sample are within the measurement error of the AFM measurements indicating chemical induced surface modification of surfaces of an implant generate nanoscale features on similar length scales when compared to line-of-sight modification processes. It should be noted, one or more features within a size of a measurement area of the AFM may influence one or more of the values provided in Table 12. Values provided in Table 12 include AFM measurements obtained using a Bruker Dimension Icon in PeakForce Tapping mode, and a Scanasyst Air probe (0.4 N/m spring constant, Bruker) was used with 0.5 to 2.5 nN setpoints and 0.25 to 1 Hz scan rates, depending on sample topography. Samples provided in Table 12 were analyzed using 500 nm image sizes and data analysis was done using Nanoscope Analysis. The Sq of the unmodified implant may range from about 3 nm to about 9 nm, about 4 nm to about 8 nm, approximately 6 nm. The Sq of the first comparative sample may range from about 6 nm to about 19 nm, about 6 nm to about 18 nm, approximately 12 nm. The Sq of the wrought implant may range about 4 nm to about 21 nm, about 4 nm to about 20 nm, approximately 12 nm. The Sq of the additively manufactured implant may range from about 3 nm to about 22 nm, about 4 nm to about 20 nm, approximately 12 nm. The Sz of the unmodified implant may range from about 29 nm to about 66 nm, approximately 47 nm. The Sz of the first comparative sample may range from about 50 nm to about 116 nm, about 75 nm to about 100 nm, approximately 83 nm. The Sz of the wrought implant may range from about 28 nm to about 150 nm, about 37 nm to 100 nm, approximately 81 nm. The Sz of the additively manufactured implant may range from about 27 nm to about 151 nm, about 50 nm to about 95 nm, approximately 90 nm. The Sa of the unmodified implant may range from about 2 nm to about 7 nm, about 2 nm to about 5 nm, approximately 4 nm. The Sa of the first comparative sample may range from about 4 nm to about 16 nm, about 5 nm to about 12 nm, approximately 10 nm. The Sa of the wrought implant may range from about 3 nm to about 17 nm, about 4 nm to about 15 nm, approximately 10 nm. The Sa of the additively manufactured implant may range from about 2 nm to about 17 nm, about 3 nm to about 17 nm, approximately 10 nm.

TABLE 12
Comparative AFM measurements of implants

| | | | | ADDITIVELY |
|---|---|---|---|---|
| | | FIRST | WROUGHT | MANUFACTURED |
| | UNMODIFIED | COMPARATIVE | MODIFIED | MODIFIED |
| | IMPLANT | SAMPLE | IMPLANT | IMPLANT |
| Sq | 5.8 ± 2.6 | 12.4 ± 6.3 | 12.6 ± 8.0 | 12.4 ± 9.0 |
| (nm) | | | | |
| Sz (nm) | 47.4 ± 18.0 | 83.5 ± 33.2 | 81.1 ± 43.5 | 89.3 ± 61.7 |
| Sa (nm) | 4.4 ± 2.1 | 10.0 ± 5.3 | 9.9 ± 6.6 | 9.8 ± 7.3 |

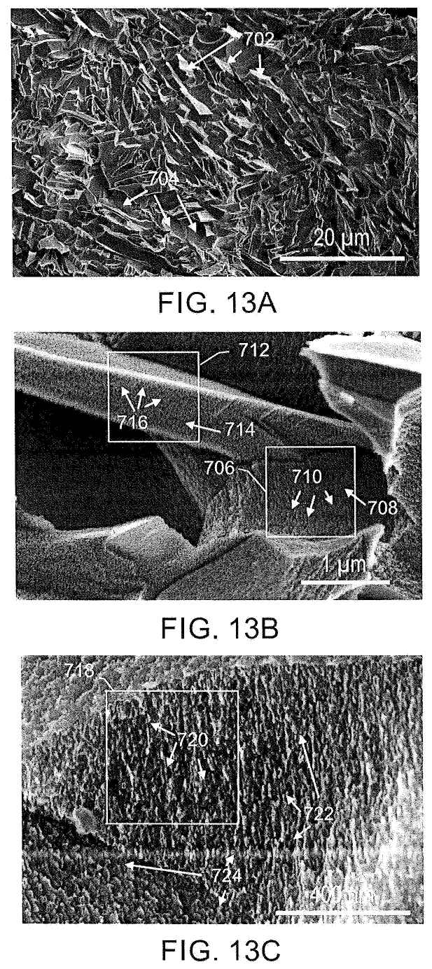

FIG. 13A is a SEM image of a portion of a modified implant generated using additive manufacturing, including a plurality of microscale structures generated by contacting an implant 12 with at least an alkaline solution 44 and a peroxide solution 56, in accordance with an embodiment of the present disclosure. The implant 12 is an implant formed from metal additive manufacturing. The additively manufactured implant is treated with at least the alkaline solution 44 and the peroxide solution 56 to form a modified additively manufactured implant. The scale bar is 20 μm. As illustrated by arrows 702, the microscale structures generate features forming needlelike structures on the surface of the portion of the modified implant 14 with microscale valleys between the needlelike structures as illustrated by arrow 704. FIG. 13B is a SEM image of a portion of the modified implant 14 of FIG. 13A, including microscale structures and nanoscale structures generated by contacting the additively manufactured implant with the alkaline solution 44 and the peroxide solution 56, in accordance with an embodiment of the present disclosure. The scale bar is 1 μm. As illustrated by a first box 706 (e.g., 1 μm by 1 μm box) a first microscale feature 708 may include nanoscale features 710. The nanoscale features 710 may form a corrugated surface morphology between the needlelike structures of the modified implant 14. Additionally, as illustrated by a second box 712 (e.g., 1 μm by 1 μm box), a second microscale feature 714 may form sharp edges on the surface of the modified implant 14 and may include a plurality of nanoscale features 716. FIG. 13C is a SEM image of a portion of the modified implant 14 of FIG. 13B at a higher magnification, including nanoscale structures generated by contacting the modified additively manufactured implant with the alkaline solution 44 and the peroxide solution 56, in accordance with an embodiment of the present disclosure. The scale bar is 400 nm. As illustrated by a first box 718 (e.g., 400 nm by 400 nm box) a plurality of nanoscale features 720 may form a corrugated surface morphology. Additionally, as illustrated by differences in contrast within the SEM image, the portion of the modified additively manufactured implant may include a first portion 722 of the plurality of nanoscale features 720 of a first conformation (e.g., electron density, sample height) and a second portion 724 of the nanoscales features 720 of a second conformation.

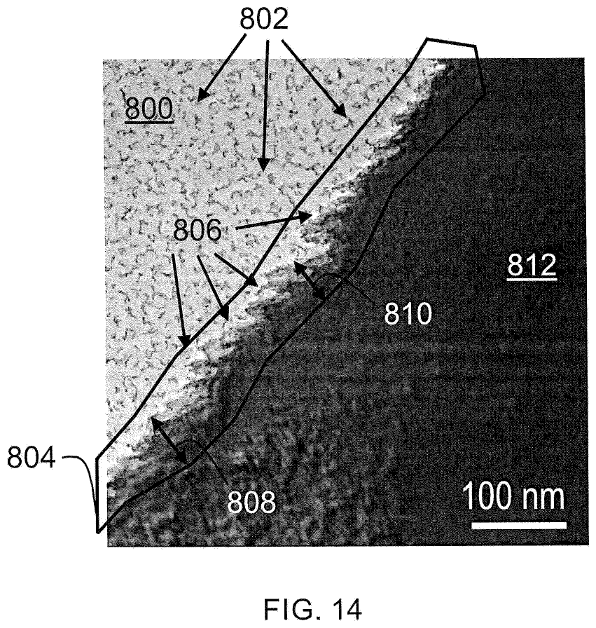

FIG. 14 is a HR-TEM image from the cross section following FIB milling of a modified additively manufactured implant including nanoscale structures, in accordance with an embodiment of the present disclosure. The modified additively manufactured implant of FIG. 14 is treated according to the process 40 as described in reference FIG. 2. The implant 12 used to form the modified additively manufactured implant is generated using additive manufacturing. The scale bar is 100 nm. A cross section of the modified additively manufactured implant is milled using FIB and imaged on a carbon protective layer 800 as shown by arrow 802. The modified additively manufactured implant includes a heterogenous surface morphology as illustrated by box 804. The heterogenous surface morphology includes a plurality of sharp edges 806. The surface morphology as illustrated by box 804 is generated based on treatment of the surface of the modified additively manufactured implant with at least the alkaline solution 44 and the peroxide solution 56. The sharp edges 806 generated by treatment of the surface forms a rough surface with a thickness from about 30 nm to 50 nm as illustrated by arrows 808 and 810. The additively manufactured implant includes a bulk portion 812 of higher electron density. The bulk portion 812 of the additively manufactured implant may include metals, metal alloys, or a combination thereof.

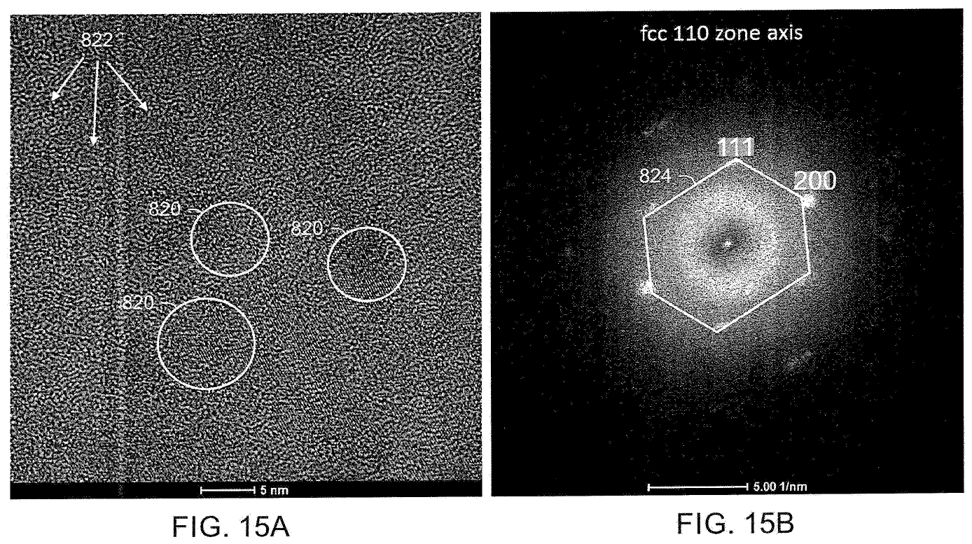

FIG. 15A is a high resolution TEM (HR-TEM) image of a modified additively manufactured implant including one or more nanocrystals 820 within an amorphous lattice 822, in accordance with an embodiment of the present disclosure. The modified additively manufactured implant of FIGS. 15A and 15B are treated according to the process 40 as described in reference FIG. 2. The scale bar is 5 nm. As shown, the modified additively manufactured implant includes the one or more nanocrystals 820 within the amorphous lattice 822 demonstrating increased ordering of the modified additively manufactured implants when compared to the modified implants 14 shown in FIG. 7. The nanocrystals 820 may correspond to an additional phase of the surface of the additively manufactured implant in addition to the α-phase (HCP) and β-phase (BCC). Without wishing to be bound by theory, the one or more nanocrystals 820 may be formed during treatment of the additive manufactured implant due to non-equilibrium processing resulting in changes in crystal lattice configurations. For example, during modification of the α-phase (HCP) and β-phase (BCC) of the additively manufactured implant one or more crystal defects may be introduced into the crystal structure due to introduction of dislocations, twins, and the like. As such, a FCC phase may be formed due to interstitial defects and/or may be stabilized through one or more additional defects introduced during chemical treatment. Additionally and/or alternatively, the FCC phase may be introduced into the modified additively manufacture implant to accommodate strain effects due to etching of the α-phase (HCP) and β-phase (BCC) with the alkaline solution 44 and/or the peroxide solution 56. FIG. 15B is a diffraction pattern of the HR-TEM image of FIG. 15A at a face centered cubic (FCC) 110 zone axis indicative of short-range ordering due to the presence of the nanocrystals 820 within the additively manufactured implant, in accordance with an embodiment of the present disclosure. Without wishing to be bound by theory, a FFC region 824 shown in the diffraction pattern is indicative of nanocrystals 820 having a FCC grain structure. Additionally, presence of multiple diffraction rings (not shown) is indicative of formation of the nanocrystals 820 with FCC grain structure within the amorphous lattice 822. In some instances, the amorphous lattice 822 may include HCP grains corresponding to an α-phase and and/or BCC grains corresponding to a β-phase.

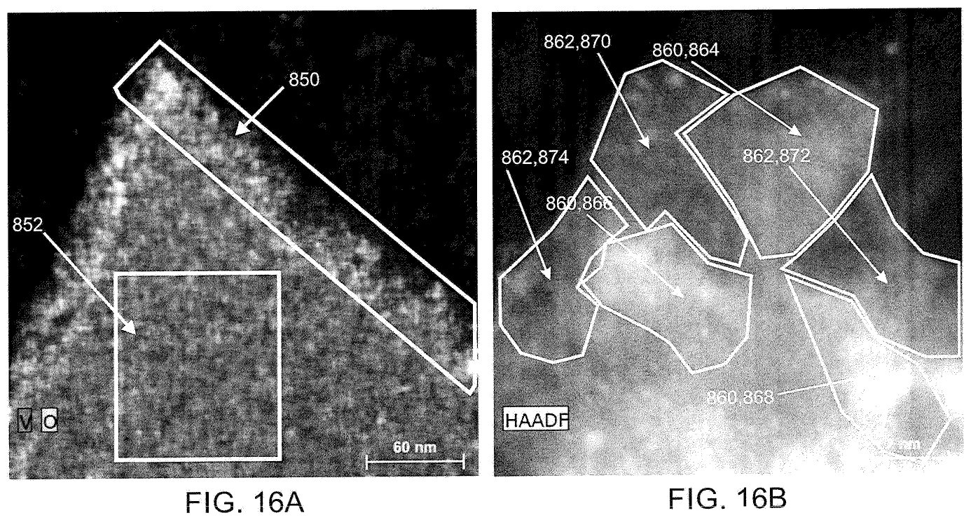

FIG. 16A is a HR-TEM image including one or more EDS imaging areas of a modified additively manufactured implant, in accordance with an embodiment of the present disclosure. The modified additively manufactured implant includes a vanadium rich grain surface. The scale bar is 60 nm. As shown, a surface oxide area 850 of the modified additively manufactured implant is rich in vanadium oxide. The surface oxide area 850 of the modified additively manufactured implant includes approximately 30 nm from an edge of the modified additively manufactured implant. A surface area 852 of the modified additively manufactured implant is rich in vanadium and titanium. Table 13 includes the elemental composition and elemental ratios of the surface oxide area 850 and the surface area 852. The surface oxide area 850 has a titanium to oxygen ratio ranging from about 1 to about 3 (e.g., about 1.41), a titanium to vanadium ratio ranging from about 3 to about 3.5 (e.g., about 3.21), a titanium to aluminum ratio ranging from about 20 to about 25 (e.g., about 22.43), and an average titanium to iron ratio ranging from about 20 to about 25 (e.g., about 24.38). The surface area 852 has a titanium to oxygen ratio ranging from about 5 to about 10 (e.g., about 8.29) a titanium to vanadium ratio ranging from about 3 to about 4 (e.g., about 3.63), a titanium to aluminum ratio ranging from about 15 to about 23 (e.g., about 20.54), and an average titanium to iron ratio ranging from about 25 to 35 (e.g., about 28.51).

TABLE 13
Elemental composition and elemental ratios of
surface oxide and surface areas of FIG. 16A

| | Area 850 | Area 852 |
|---|---|---|
| Atom C. [at %] ± 3σ | | |
| V | 14.82 ± 2.15 | 18.61 ± 2.01 |
| Al | 2.12 ± 0.35 | 3.29 ± 0.29 |
| Ti | 47.55 ± 5.82 | 67.58 ± 6.55 |
| O | 33.57 ± 1.60 | 8.15 ± 0.39 |
| Fe | 1.95 ± 0.55 | 2.37 ± 0.40 |
| Elemental Ratios | | |
| Ti/O | 1.41 | 8.29 |
| Ti/V | 3.21 | 3.63 |
| Ti/Al | 22.43 | 20.54 |
| Ti/Fe | 24.38 | 28.51 |

FIG. 16B is a HR-TEM image including one or more EDS imaging areas of a modified additively manufactured implant at an increased magnification, in accordance with an embodiment of the present disclosure. The scale bar is 7 nm. The modified additively manufactured implant includes one or more nanocrystalline areas 860 and one or more amorphous areas 862. The one or more nanocrystalline areas 860 includes a first nanocrystalline area 864, a second nanocrystalline area 866, and a third nanocrystalline area 868. The one or more amorphous areas 862 include a first amorphous area 870, a second amorphous area 872, and a third amorphous area 874. Table 14 includes the elemental composition and elemental ratios of the nanocrystalline areas 860 and the amorphous areas 862. The nanocrystalline areas 860 have a titanium to oxygen ratio ranging from about 0.70 to about 0.82, with an average of about 0.76, a titanium to vanadium ratio ranging from about 25.5 to about 58.0, with an average of about 37.05, a titanium to aluminum ratio ranging from about 6.5 to about 11.0, with an average of about 8.77, and a titanium to iron ratio ranging from about 30.0 to about 70.0, with an average of about 49.89. The amorphous areas 862 have a titanium to oxygen ratio ranging from about 0.55 to about 0.69, with an average of about 0.64, a titanium to vanadium ratio ranging from about 14.0 to about 45.0, with an average of about 27.77, a titanium to aluminum ratio ranging from about 4.0 to about 13.0, with an average of about 8.35, and a titanium to iron ratio ranging from about 20 to about 57, with an average of about 41.57.

TABLE 14
Elemental composition and elemental ratios of nanocrystalline areas and amorphous areas of FIG. 16B

| | Area 864 | Area 866 | Area 868 | Area 870 | Area 872 | Area 874 |
|---|---|---|---|---|---|---|
| Atom C. | | | | | | |
| [at %] ± 3σ | | | | | | |
| V | 1.51 ± 1.20 | 0.73 ± 0.79 | 1.39 ± 1.04 | 2.57 ± 2.50 | — | 0.84 ± 1.45 |
| Al | 3.81 ± 1.23 | 5.77 ± 1.43 | 4.53 ± 1.23 | 2.90 ± 1.71 | 4.87 ± 1.72 | 7.35 ± 2.73 |
| Ti | 40.12 ± 7.94 | 41.11 ± 7.80 | 39.28 ± 7.51 | 36.60 ± 10.17 | 37.49 ± 8.71 | 34.70 ± 9.93 |
| O | 53.24 ± 4.45 | 51.58 ± 4.13 | 54.23 ± 4.23 | 56.16 ± 6.73 | 56.87 ± 5.51 | 56.50 ± 6.80 |
| Fe | 1.31 ± 1.23 | 0.82 ± 0.91 | 0.57 ± 0.75 | 1.77 ± 2.28 | 0.78 ± 1.18 | 0.62 ± 1.37 |
| Elemental | | | | | | |
| Ratios | | | | | | |
| Ti/O | 0.75 | 0.80 | 0.72 | 0.65 | 0.66 | 0.61 |
| Ti/V | 26.57 | 56.32 | 28.26 | 14.24 | — | 41.30 |
| Ti/Al | 10.53 | 7.12 | 8.67 | 12.62 | 7.69 | 4.73 |
| Ti/Fe | 30.63 | 50.13 | 68.91 | 20.68 | 48.06 | 55.97 |

### Osteogenic Markers on MSCs Grown on Surfaces of Modified Implants

Osteogenic differentiation is a continuous process characterized by increased and decreased presence of several proteins such as alkaline phosphatase (ALP) and/or osteocalcin (OCN). Additional and/or alternative protein expression represent lifetime osteogenic markers, such as osteopontin (OPN), which continue to increase with time as cells mature to an osteoblast phenotype. Osteoblast differentiation begins with mesenchymal stem cells (MSCs) progressing to an intermediate progenitor capable of undergoing either osteogenesis or chondrogenesis and expressing ALP. Intermediate progenitors that commit to an osteogenic lineage (e.g., preosteoblasts), increase the expression of ALP, and start expressing the extracellular matrix protein OPN. As the preosteoblast progresses to an osteoblast expression of OPN continues to increase and expression of OCN is increased. The osteoblast will in time mature further and begin transitioning to an osteocyte or undergo apoptosis. The mature osteoblast state is characterized by a decrease in ALP and once the osteoblast differentiates to an osteocyte the expression of OCN may also decrease. OPN expression is detected throughout osteogenic lifetime from a preosteoblast to an osteocyte. Sclerostin (SOST) is a secreted protein that regulates bone remodeling and is produced by terminally differentiated osteocyte. Proteins expressed by osteoblasts work in concert to produce an osteoid matrix of Type I collagen network in the presence of increased phosphate ions that mineralize. Without wishing to be bound by theory, mineralization may be catalyzed by the early bone marker ALP. The mineralization of synthesized osteoid deposited by the osteoblasts leads to bone tissue formation. Surface texture and/or surface chemistry may influence one or multiple steps in the osteoblast differentiation and mineralization process.

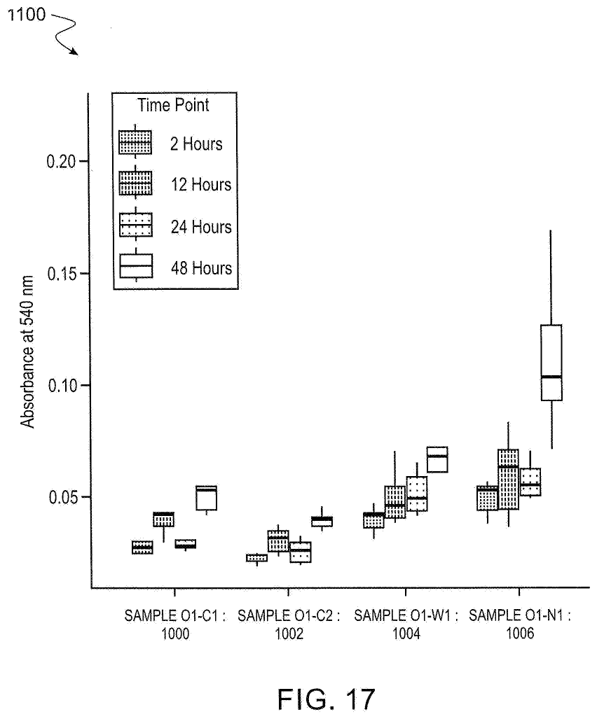

FIGS. 17-22 illustrate cellular outcomes of osseointegration for wrought modified implants with microfeatures and nanofeatures and comparative samples. As demonstrated in FIGS. 17-22, and discussed in more detail below, expression of various markers is higher for the modified implant 14 as compared implants that do not include the microfeatures and nanofeatures described herein.

FIGS. 17-22 include cellular outcomes for various surfaces including Sample O1-C1 1000, Sample O1-C2 1002, Sample O1-W1 1004, and Sample O1-N1 1006. Sample O1-C1 1000 corresponds to a machined implant (e.g., Ti implant). Sample O1-C2 1002 corresponds to an implant modified using line-of-sight subtractive surface modification. Sample O1-W1 1004 corresponds to a modified implant 46 (e.g., a treated wrought implant). Sample O1-N1 1006 corresponds to a modified additively manufactured implant (e.g., a treated additively manufactured implant).

Cell adhesion outcomes outlined illustrated in FIG. 17 are conducted on one or more discs from each sample group, seeded with human MSCs at 1,000 cells/cm² and maintained in basal media composed of: αMEM with 10% fetal bovine serum (FBS) and 1% penicillin/streptomycin (P/S). At 2 h, 6 h, 12 h and 24 h post seeding samples were washed with phosphate-buffered saline (PBS) and mechanically challenged on an orbital shaker for about 5 minutes. After mechanical challenge, samples were fixed with 3.7 paraformaldehyde and stained with 0.05% crystal violet in deionized (DI) water. Crystal violet was then solubilized in methanol and absorbance measured at 540 nm for quantification of cell number.

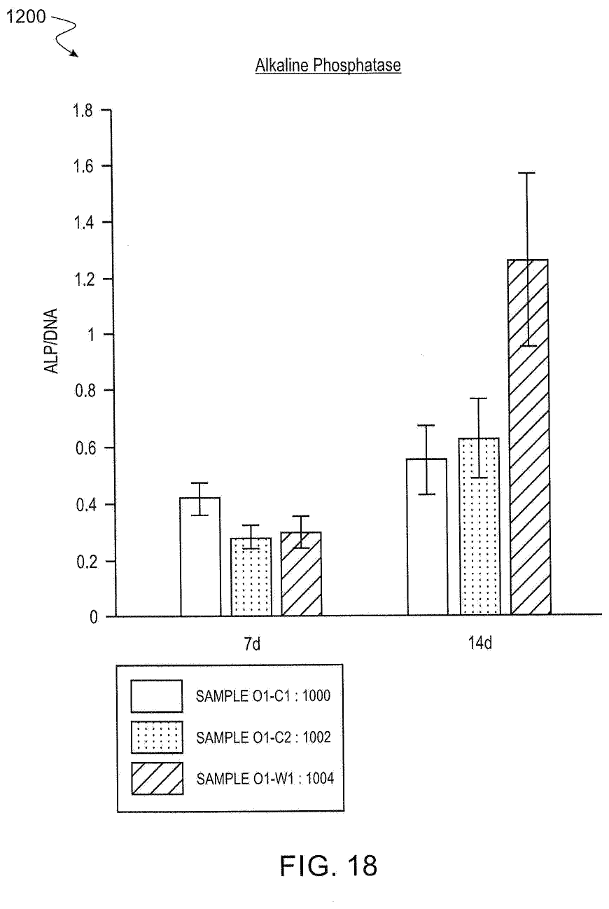

Osteogenic differentiation protein assay outcomes illustrated in FIGS. 18-20, 23, and 24 are conducted on one or more discs from each sample group, seeded with human MSCs at 10,000 cells/cm² and maintained in culture media composed of: αMEM with 10% FBS and 1% P/S, 3 mM beta-glycerophosphate and 50 μg/mL ascorbic acid. At day 3, day 7, and day 14 post seeding samples were lysed in 200 μL of tissue protein extraction reagent (T-PER) and transferred to −80 °C for storage. ALP was assayed with an enzymatic p-nitrophenol reaction. OCN, OPN and SOST protein levels in the media were quantified using associated enzyme-linked immunosorbent assays (ELISAs) according to the manufacturer's protocols.

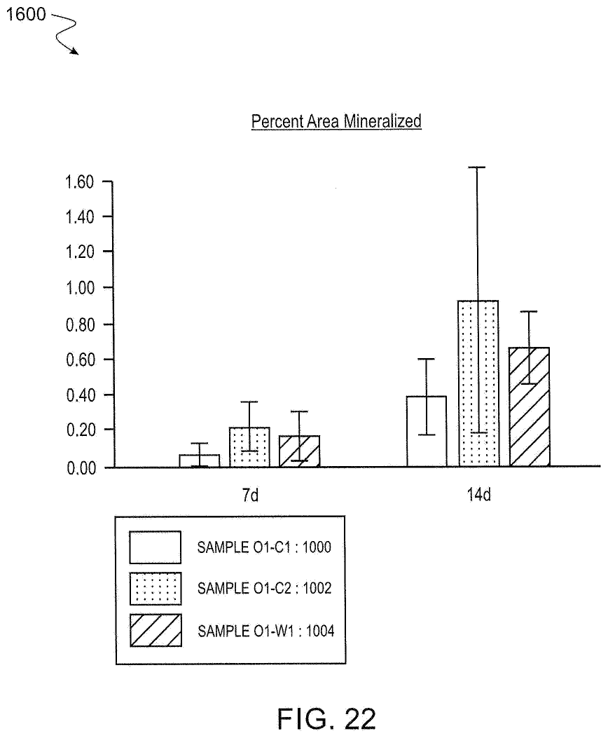

Mineralized tissue outcomes illustrated in FIG. 22 are conducted on one or more discs from each sample group, seeded with human MSCs at 10,000 cells/cm² and maintained in culture media composed of: αMEM with 10% FBS and 1% P/S, 3 mM beta-glycerophosphate and 50 μg/mL ascorbic acid. Prior to staining, the discs were incubated in a 20 μM solution of tetracycline for 24 h to fluorescently label calcium deposits. At day 7 and day 14 post seeding samples were fixed with 3.7% paraformaldehyde. Microscopy images were acquired with a Leica DM5500B and processed with Leica LASX imaging software. Mineralized nodules were quantified and compared to total surface area.

FIG. 17 is a graph 1100 including crystal violet absorbance to characterize cell numbers on the modified implant 12 in comparison to comparative samples, in accordance with an embodiment of the present disclosure. The graph 1100 illustrates differences in crystal violet absorbance indicative of cell numbers at four time points: 2 hours, 12 hours, 24 hours and 48 hours. As shown, Sample O1-W1 1004 and Sample O1-N1 1006 increase cell attachment and proliferation from 2 h to 48 h post seeding when compared with Sample O1-C1 1000 and Sample O1-C2 1002.

FIG. 18 is a graph 1200 of alkaline phosphate (ALP) expression in a modified implant in comparison to comparative samples, in accordance with an embodiment of the present disclosure. The graph 1200 shows the ALP expression per DNA at two time points: 7 days, and 14 days. As shown, ALP expression significantly increases from day 7 to day 14 for modified implant surfaces (e.g., Sample O1-W1 1004). Further, ALP expression of the modified implant is significantly higher for Sample O1-W1 1004 as compared to Sample O1-C1 1000 and Sample O1-C2 1002 (e.g., machined or grit blast/acid etched surfaces) at day 14.

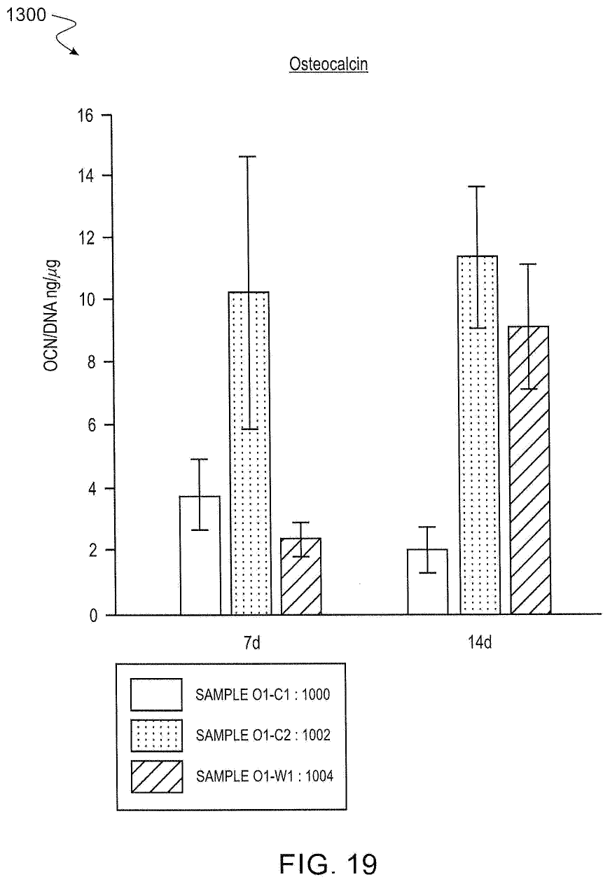

FIG. 19 is a graph 1300 of osteocalcin (OCN) expression of a modified implant in comparison to comparative samples, in accordance with an embodiment of the present disclosure. The graph 1300 shows the OCN expression (ng/μg DNA) at two time points: 7 days and 14 days. As shown, OCN expression of Sample O1-C2 1002 and Sample O1-W1 1004 significantly increase from day 7 to per day 14 (e.g., implants with treated surfaces). Further, OCN expression of Sample O1-W1 1004 (e.g., the modified implant 14) is significantly higher than Sample O1-C1 1000 (e.g., machined implant) at day 14.

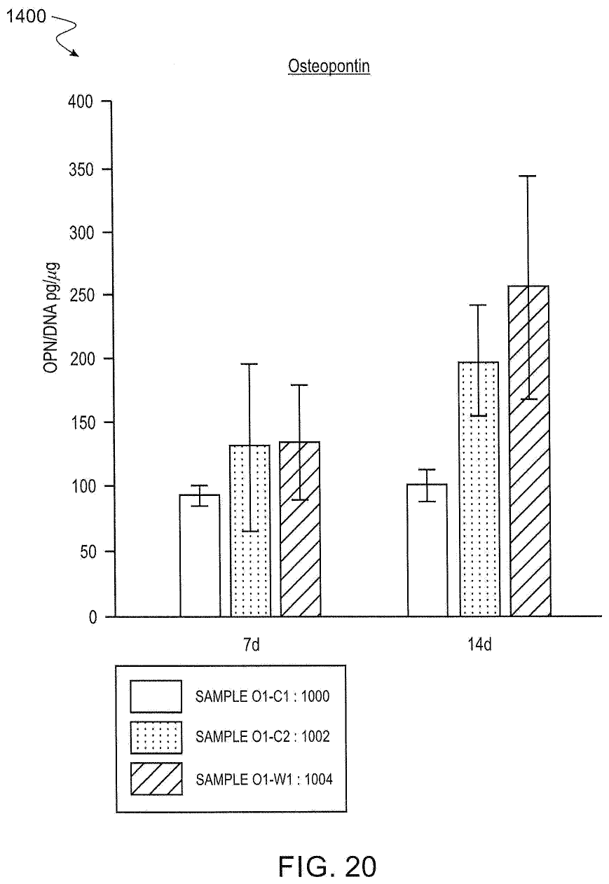

FIG. 20 is a graph 1400 of osteopontin (OPN) expression in a modified implant in comparison to comparative samples, in accordance with an embodiment of the present disclosure. The graph 1400 shows the OPN expression (pg/μg per DNA) at two time points: 7 days and 14 days. OPN levels is a lifetime differentiation maker and OPN expression continues to rise as MSCs mature to osteoblast and/or osteocytes. As shown, OPN expression of Sample O1-C2 1002 and Sample O1-W1 1004 significantly increases from day 7 to day 14 (e.g., implants with treated surfaces). Further, OPN expression of Sample O1-C1 1004 (e.g., the modified implant 14) is significantly higher than Sample O1-C1 1000 (e.g., machined implant) at day 14.

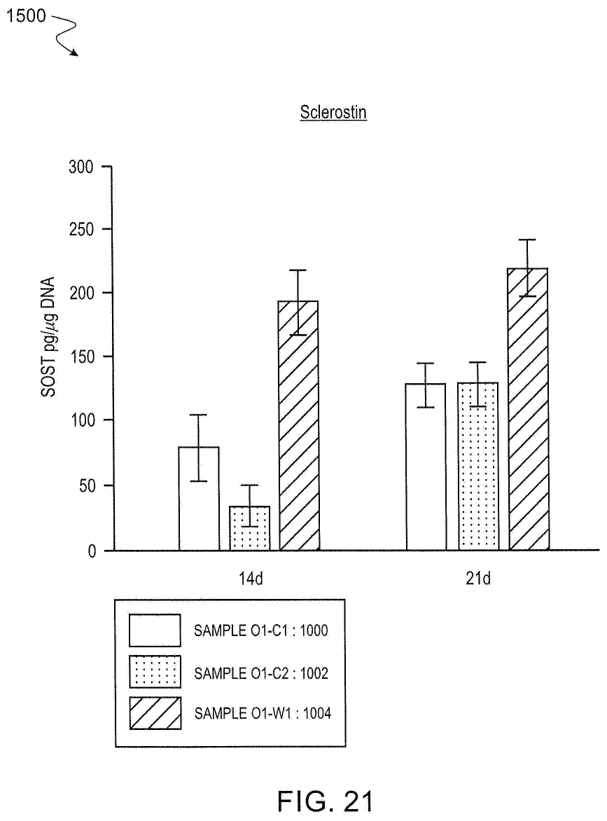

FIG. 21 is a graph 1500 of sclerostin (SOST) expression of a modified implant in comparison to comparative samples, in accordance with an embodiment of the present disclosure. The graph 1500 shows the SOST expression (pg/ug per DNA) at two time points: 14 days and 21 days. SOST expression continues to rise as MSCs mature to osteoblasts and/or osteocytes. As shown, SOST expression of Sample O1-C1 1000, Sample O1-C2 1002, and Sample O1-W1 1004 increases from day 14 to day 21. Further, SOST expression of Sample O1-C1 1004 (e.g., the modified implant 14) is significantly higher than Sample O1-C1 1000 (e.g., machined implant) and Sample O1-C2 1002 (e.g., grit blast/acid etched surfaces) at day 14 and day 21.

FIG. 22 is a graph 1600 of mineralization expression in a modified implant in comparison to comparative samples, in accordance with an embodiment of the present disclosure. The graph 1600 shows the percent of area mineralized increases from day 7 to day 14 for modified implant surfaces and comparative samples (e.g., Sample O1-C1 1000, Sample O1-C2 1002, and Sample O1-W1 1004). The percent of area with mineralized tissue of d Sample O1-W1 1004 is significantly higher than Sample O1-C1 1000 at day 14 Without wishing to be bound by theory, the microstructures and nanostructures on the surface of Sample O1-W1 1004 (e.g., the modified implant 14) improve osseointegration of bone-contacting surfaces of the modified implant 14.

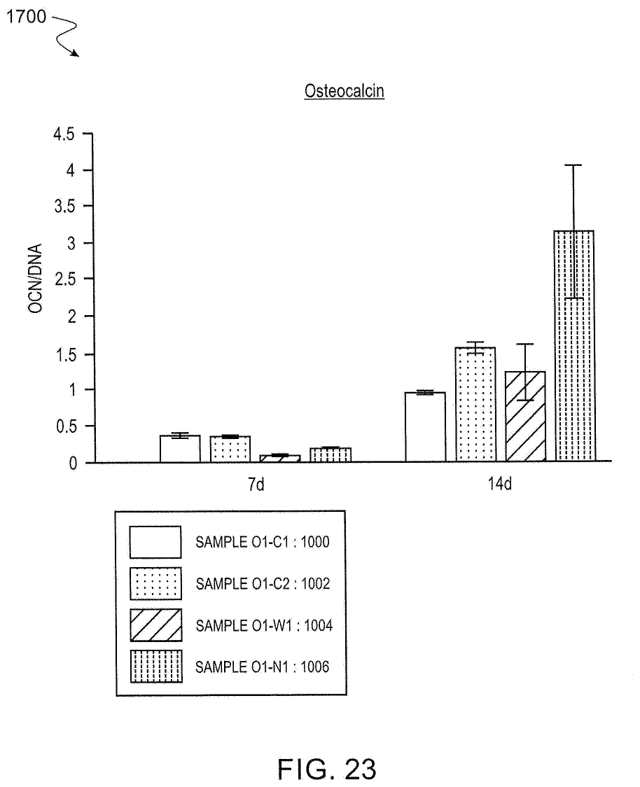

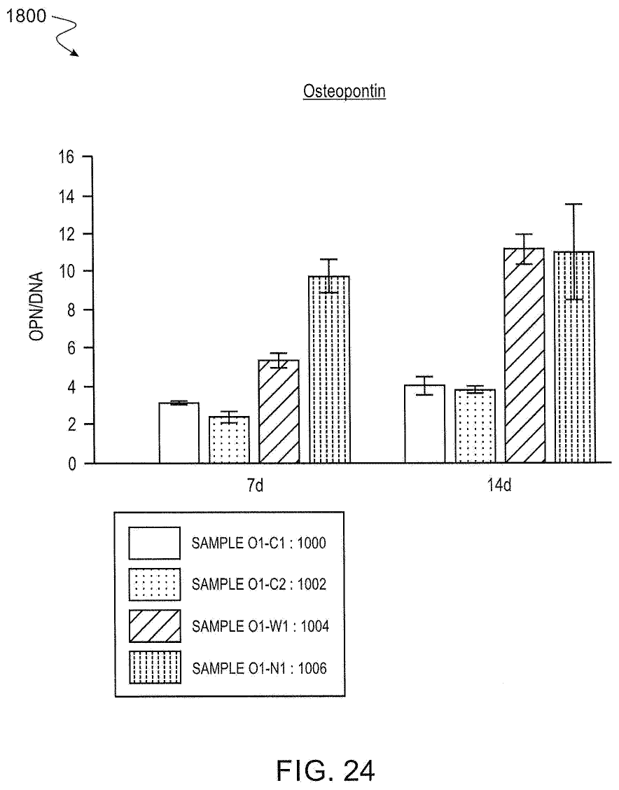

FIGS. 23 and 24 illustrate cellular outcomes of osseointegration for wrought modified implants and additively manufactured modified implants with microfeatures and nanofeatures and comparative samples.

FIG. 23 is a graph 1700 of osteocalcin (OCN) expression of a wrought modified implant and an additively manufactured modified implant in comparison to comparative samples, in accordance with an embodiment of the present disclosure. The graph 1700 includes OCN expression of Sample O1-C1 1000, Sample O1-C2 1002, Sample O1-W1 1004, and Sample O1-N1 1006. The graph 1700 illustrates the OCN expression (ng/μg per DNA) at two time points: 7 days and 14 days. As shown, OCN expression significantly increases from day 7 to day 14 for Sample O1-C1 1000, Sample O1-C2 1002, Sample O1-W1 1004, and Sample O1-N1 1006 (e.g., modified implant surfaces and comparative groups). OCN expression of the Sample O1-C2 1002 and Sample O1-W1 1004 is higher than Sample O1-C1 1000 at day 14 and Sample O1-N1 1006 (e.g., the additively manufactured modified implant) significantly increased OCN expression at day 14.

FIG. 24 is a graph 1800 of osteopontin (OPN) expression of a wrought and of an additive modified implant in comparison to comparative samples, in accordance with an embodiment of the present disclosure. The graph 1800 includes OCN expression of Sample O1-C1 1000, Sample O1-C2 1002, Sample O1-W1 1004, and Sample O1-N1 1006. The graph 1800 illustrates the OPN expression (pg/μg per DNA) at two time points: 7 days and 14 days. OPN level is a lifetime differentiation maker and OPN expression continues to rise as MSCs mature to osteoblast and/or osteocytes. As shown, OPN expression significantly increases from day 7 to day 14 for both Sample O1-W1 1004 and Sample O1-N1 1006 (e.g., the wrought modified implant and additively manufactured modified implant surfaces). Further, OPN expression for both Sample O1-W1 1004 and Sample O1-N1 1006 (e.g., the wrought modified implant and additively manufactured modified implant surfaces) is significantly higher than Sample O1-C1 1000 and Sample O1-C2 1002 (e.g., machined and grit blast/acid etched surfaces) at day 14.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.”

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method for modifying a surface of an implantable device, the method comprising:
contacting the implantable device with an alkaline etch solution comprising a chelating agent, wherein the alkaline etch solution etches an alpha phase and a beta phase of the surface of the implantable device; and
contacting the implantable device with a hydrogen peroxide solution, wherein the hydrogen peroxide solution further etches the beta phase of the surface of the implantable device.

2. The method of claim 1, comprising contacting the implantable device with the hydrogen peroxide solution subsequent to contacting the implantable device with the alkaline etch solution.

3. The method of claim 1, wherein the chelating agent comprises a gluconate salt or gluconic acid.

4. The method of claim 1, comprising performing one or more cleaning steps configured to remove insoluble precipitates, soluble salts, metal precipitates, unbound metal oxides, or a combination thereof from the surface of the implantable device.

5. The method of claim 1, wherein the alkaline etch solution comprises 0.1 to 10 wt % of the chelating agent.

6. The method of claim 1, further comprising contacting the implantable device with an acid solution, wherein the acid solution comprises nitric acid, citric acid, or both.

7. The method of claim 1, wherein the hydrogen peroxide solution further etches the alpha phase of the surface of the implantable device.

8. The method of claim 1, wherein the hydrogen peroxide solution comprises between 2 to 20% by volume of hydrogen peroxide.

9. The method of claim 1, comprising contacting the implantable device with the alkaline etch solution at a reaction temperature between 80 °C to 95 °C for 5 to 30 minutes (mins).

10. The method of claim 1, comprising contacting the implantable device with the hydrogen peroxide solution at a reaction temperature between 40 °C to 60 °C for 1 to 20 minutes (mins).

11. A method for forming one or more microstructures, one or more nanostructures, or a combination thereof on a surface of an implantable device, the method comprising:
etching an alpha phase and a beta phase of the surface of the implantable device; and
further etching the beta phase of the surface of the implantable device, wherein etching the beta phase of the surface of the implantable device comprises reducing a vanadium content of the etched surface of the implantable device.

12. The method of claim 11, further comprising removing one or more alloying elements from the etched surface of the implantable device.

13. The method of claim 11, comprising etching the alpha phase and the beta phase using an alkaline etch solution comprising a metal hydroxide and a chelating agent.

14. The method of claim 13, wherein the chelating agent comprises a gluconate salt or gluconic acid.

15. The method of claim 11, comprising reducing the vanadium content of the etched surface of the implantable device using a hydrogen peroxide solution.

16. A method for modifying a surface of an implantable device, the method comprising:
contacting the implantable device with an alkaline etch solution under a first set of reaction conditions to form an alkaline etched implantable device, wherein the alkaline etch solution comprises a metal hydroxide and a chelating agent, wherein the alkaline etch solution etches an alpha phase of the surface of the implantable device
contacting the alkaline etched implantable device with an acid solution to form an acid etched implantable device; and
contacting the acid etched implantable device with a hydrogen peroxide solution under a second set of reaction conditions to form a peroxide etched implantable device, wherein the hydrogen peroxide solution etches a beta phase of the surface of the implantable device, wherein the first set of reaction conditions and the second set of reaction conditions are different.

17. The method of claim 16, wherein the alkaline etch solution etches the beta phase of the surface of the implantable device.

18. The method of claim 16, wherein the first set of reaction conditions comprises a first reaction temperature between 80 °C to 95 °C, and wherein the second set of reaction condition comprises a second reaction temperature between 40 °C to 60 °C.

19. The method of claim 16, further comprising performing one or more cleaning steps configured to remove insoluble precipitates, soluble salts, metal precipitates, unbound metal oxides, or a combination thereof from the alkaline etched implantable device, the peroxide etched implantable device, or both.

20. The method of claim 16, wherein the hydrogen peroxide solution further etches the alpha phase of the surface of the implantable device.
